(19) United States
(12) Patent Application Publication
(10) Pub. No.: US 2025/0288664 A1
(43) Pub. Date: Sep. 18, 2025

(54) COMPOSITIONS AND METHODS FOR EYE DISEASES

(71) Applicant: BEIJING SIGHTNOVO MEDICAL TECHNOLOGY CO., LTD, Beijing (CN)

(72) Inventors: Chan ZHAO, Beijing (CN); Chaoran XIA, Beijing (CN); Weixing ZHONG, Beijing (CN); Miao ZHANG, Beijing (CN)

(21) Appl. No.: 18/861,179

(22) PCT Filed: Apr. 27, 2023
(86) PCT No.: PCT/CN2023/091039

§ 371 Date: Oct. 28, 2024

(30) Foreign Application Priority Data
Apr. 27, 2022 (WO) PCT/CN2022/089533

(51) Int. Cl. A61K 39/395; A61K 38/17; A61K 45/06; A61P 27/02; C07K 16/22

(52) U.S. Cl. CPC A61K 39/3955; A61K 38/179; A61K 45/06; A61P 27/02; C07K 16/22; C07K 2317/24

(57) ABSTRACT

It relates to methods and compositions for determining anatomical response to an VEGF antagonist treatment in a subject's eye, comprising determining a VEGF level in the aqueous humor of the subject or in combination with determining a bl-CMT level in the subject, wherein a determined VEGF level higher than a VEGF cut-off value/range or in combination with a determined bl-CMT level higher than a bl-CMT cut off value/range indicate that the eye has a good anatomical response to intravitreal, suprachoroidal, and/or subretinal VEGF antagonist treatment, wherein a determined VEGF level lower than the VEGF cut-off value/range, or a determined bl-CMT level lower than the bl-CMT cut off value/range indicates that the eye has a poor anatomical response to intravitreal, suprachoroidal, and/or subretinal VEGF antagonist treatment. The aqueous humor VEGF level and/or the bl-CMT level can be used as a companion diagnostic to predict responsiveness of the subject to anti-VEGF therapies.

### FIELD OF THE INVENTION

The present disclosure relates in some aspects to methods and compositions for treatment of retinal vein occlusion and companion diagnostics.

### BACKGROUND OF THE INVENTION

Retinal vein occlusion (RVO) is one of the most common retinal vascular diseases [1, 2]. It is caused by partial or complete occlusion of venous blood flow which leads to an increase in venous pressure with subsequent leakage of the retinal microvasculature behind the occlusion site [3]. Improved methods for treating RVO and symptoms thereof are needed. Provided herein are methods and compositions that address such and other needs.

RVO can include blockage of the main retinal vein called central retinal vein occlusion (CRVO), and/or a smaller vein called branch retinal vein occlusion (BRVO). The pathological mechanisms of RVO are a series of changes that occur after retinal vein occlusion, including hypoxia, increased vascular permeability, inflammation, and neovascularization, and involves a complex interplay among a variety of vascular and inflammatory mediators.

Vascular endothelial growth factor (VEGF), a potent mediator of angiogenesis and vascular permeability, is a key molecule involved in the pathophysiological processes in RVO [4, 5], and intravitreal anti-VEGF agents is currently considered as the first line treatment for macular edema (ME) secondary to RVO (RVO-ME) [6]. On the other hand, however, a substantial proportion of eyes with RVO-ME respond insufficiently to anti-VEGF agents, and notably, they generally respond well to intravitreal glucocorticosteroids with visual outcome negatively correlated to disease duration [7]. These findings suggest that RVO-ME could be clustered into two endotypes, one in which VEGF plays a central role and therefore responds well to intravitreal anti-VEGF agents (tentatively referred to as the “anti-VEGF responsive endotype”), and the other is less related to VEGF and has a poor response to anti-VEGF agents (tentatively referred to as the “anti-VEGF resistant endotype”). Prediction of treatment response to anti-VEGF agents at baseline may avoid unnecessary anti-VEGF treatments in the later endotype and improve the overall visual outcome of RVO-ME.

A companion diagnostic is a set of diagnostic tests that predict safety and/or effectiveness of a particular treatment, and has been increasingly recognized as a means to improve preciseness of treatments in cancer [8]. The pathophysiological therapeutic target itself is usually an ideal companion biomarker for treatments directed against the target. Supportively, Modi, A., et al described a bespoke treatment approach in which intravitreal drugs were customized according to aqueous humor cytokine levels (VEGF, interleukin (IL) 6 and IL-8) and were proved to be beneficial for an intractable RVO-ME case [9]. A few earlier studies in the literature have addressed the association between intraocular VEGF level and response to anti-VEGF agents. Campochiaro et al. reported that baseline aqueous VEGF level was inversely correlated to visual acuity (VA) improvement after 3 monthly IVR injections [10]. Similarly, Park, S. P. et al. detected higher baseline aqueous VEGF levels in patients who were unresponsive to a single IVB injection [11]. These results, however, were contradictory to the general rule that upregulation of a pathogenic molecule is usually associated with a favorable response to treatments targeting this molecule.

A comprehensive review on intraocular biomarkers for RVO was performed, which revealed that a panel of cytokines including VEGF, IL-6, IL-8, IL-10, monocyte chemoattractant protein-1 (MCP-1), interferon-gamma-inducible protein 10 (IP-10) etc. may serve as potential companion biomarkers for anti-VEGF treatment [12]. The invention disclosed herein addresses whether aqueous biological biomarkers and imaging biomarkers, individually or in combination, have predictive value on response to intravitreal anti-VEGF agent treatments.

### SUMMARY OF THE INVENTION

To evaluate whether aqueous biological biomarkers and imaging biomarkers, individually or in combination, have predictive value on response to intravitreal anti-VEGF agent treatments, the present disclosure in some aspects provides method(s) of predicting anatomical response to an anti-VEGF therapeutic agent treatment in an eye of a subject. In some embodiments, provides herein is a use of an anti-VEGF therapeutic agent for the manufacture of a medicament for treating retinal vein occlusion (RVO) in a subject, wherein a level of VEGF in the aqueous humor of the subject is higher than a cut-off value, and the cut-off value is between 10 and 100 μg/mL. In some embodiments, provided herein is a use of an anti-VEGF therapeutic agent for the manufacture of a medicament for treating retinal vein occlusion (RVO) in a subject, wherein a level of VEGF in the aqueous humor of the subject is higher than a VEGF cut-off value, and the VEGF cut-off value is between 10 and 100 μg/mL; and wherein a level of bl-CMT in the subject is higher than a bl-CMT cut-off value, and the bl-CMT cut-off value is between about 250 and about 600 μm. In some embodiments, the anti-VEGF therapeutic agent is a VEGF antagonist, which can be any therapeutic modality, e.g., antibodies, peptide or protein therapeutics, gene therapies, nucleic acids, small molecules, or any combination thereof.

In some aspect, the present invention provides a method of treating retinal vein occlusion (RVO) in a subject, comprising administrating an effective amount of an anti-VEGF therapeutic agent to the subject, wherein a level of VEGF in the aqueous humor of the subject is higher than a VEGF cut-off value, and the VEGF cut-off value is between 10 and 100 μg/mL.

In one aspect, the present invention provides the method of treating RVO in a subject as disclosed herein, wherein the anti-VEGF therapeutic agent comprises:
an anti-VEGF antibody or antigen binding fragment thereof, optionally wherein the antigen binding fragment is Fab, Fab′, F(ab′)2, or Fv;
a VEGF receptor (VEGFR) fusion protein, optionally wherein the VEGFR fusion protein is a VEGFR-Fc immunoadhesin; or
an anti-VEGF gene therapy agent, optionally wherein the anti-VEGF gene therapy agent comprises a nucleic acid encoding a VEGF antagonist, a nucleic acid encoding a protein that downregulates VEGF/VEGFR expression, and/or a nucleic acid targeting nucleic acids and/or proteins involved in VEGF/VEGFR expression, optionally wherein the anti-VEGF gene therapy agent comprises a viral or non-viral vector, optionally wherein the viral vector is an adeno-associated virus (AAV) vector.

In a further aspect, the present invention provides the method of treating RVO in a subject as disclosed herein, wherein the anti-VEGF therapeutic agent is administered or prepared to be administered intravitreally, suprachoroidally, and/or subretinally.

In an additional aspect, the present invention provides the method of treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In a further aspect, the present invention provides the method of treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In an additional aspect, the present invention provides the method of treating RVO in a subject as disclosed herein, wherein the subject has been treated with the anti-VEGF therapeutic agent or another anti-VEGF therapeutic agent.

In a further aspect, the present invention provides the method of treating RVO in a subject as disclosed herein, wherein the subject has not been treated with the anti-VEGF therapeutic agent or another anti-VEGF therapeutic agent.

In an additional aspect, the present invention provides the method of treating RVO in a subject as disclosed herein, wherein the subject has been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In a further aspect, the present invention provides the method of treating RVO in a subject as disclosed herein, wherein the subject has not been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In an additional aspect, the present invention provides the method of treating RVO in a subject as disclosed herein, wherein the subject has been treated with a steroid, optionally wherein the steroid is a corticosteroid, and optionally wherein the corticosteroid is a glucocorticosteroid.

In a further aspect, the present invention provides the method of treating RVO in a subject as disclosed herein, wherein the subject has not been treated with a steroid, optionally wherein the steroid is a corticosteroid, and optionally wherein the corticosteroid is a glucocorticosteroid.

In an additional aspect, the present invention provides the method of treating RVO in a subject as disclosed herein, wherein the VEGF cut-off value is between 30 and 90 μg/mL.

In a further aspect, the present invention provides the method of treating RVO in a subject as disclosed herein, wherein the VEGF cut-off value is about 35, about 40, about 45, about 50, about 55, about 60, about 65, about 70, about 75, about 80, or about 85 μg/mL.

In an additional aspect, the present invention provides the method of treating RVO in a subject as disclosed herein, wherein the subject has an anatomical responsiveness to the anti-VEGF therapeutic agent as measured by change of central macular thickness (CMT) or central foveal thickness (CFT), wherein the CMT or CFT is defined as the vertical distance between the internal limiting membrane and the retinal pigment epithelium at the foveal center and is also described as central foveal thickness (CFT) in some circumstances.

In a further aspect, the present invention provides the method of treating RVO in a subject as disclosed herein, wherein the subject has an anatomical responsiveness to the anti-VEGF therapeutic agent as measured by relative change of central macular thickness (CMT), wherein the relative change of CMT is defined by change of CMT divided by baseline central macular thickness (bl-CMT).

In an additional aspect, the present invention provides the method of treating RVO in a subject as disclosed herein, wherein the subject has a level of baseline central macular thickness (bl-CMT) higher than a bl-CMT cut-off value, wherein the bl-CMT cut-off value is between about 250 and about 600 μm.

In a further aspect, the present invention provides a method of treating retinal vein occlusion (RVO) in a subject as disclosed herein, comprising:
(a) determining the level of VEGF in the aqueous humor collected from the subject and the bl-CMT level in the subject, wherein the determined VEGF level is higher than the VEGF cut-off value and the determined bl-CMT level is higher than the bl-CMT cut-off value,
(b) administrating an effective amount of an anti-VEGF therapeutic agent to the subject.

In an additional aspect, the present invention provides the method of treating RVO in a subject as disclosed herein, wherein the VEGF cut-off value is between 10 and 100 μg/mL.

In a further aspect, the present invention provides the method of treating RVO in a subject as disclosed herein, wherein the bl-CMT cut-off value is between about 250 and about 600 μm.

In an additional aspect, the present invention provides the method of treating RVO in a subject as disclosed herein,

In a further aspect, the present invention provides the method of treating RVO in a subject as disclosed herein, wherein the anatomical response to the anti-VEGF therapeutic agent is measured by change of CMT which is defined as the vertical distance between the internal limiting membrane and the retinal pigment epithelium at the foveal center.

In an additional aspect, the present invention provides the method of treating RVO in a subject as disclosed herein, wherein the anatomical response to the anti-VEGF therapeutic agent is measured by relative change of CMT, wherein the relative change of CMT is defined by change of CMT divided by bl-CMT.

In a further aspect, the present invention provides the method of treating RVO in a subject as disclosed herein, wherein the determined VEGF level higher than a VEGF cut-off value of between 10 and 100 μg/mL and the determined bl-CMT level higher than a bl-CMT cut-off value of between about 250 and about 600 μm indicate that the eye has a good anatomical response to the anti-VEGF therapeutic agent treatment.

In an additional aspect, the present invention provides the method of treating RVO in a subject as disclosed herein, wherein the determined VEGF level higher than the VEGF cut-off value and the determined bl-CMT level higher than the bl-CMT cut-off value predict a change of CFT<−200 μm.

In a further aspect, the present invention provides the method of treating RVO in a subject as disclosed herein, wherein the VEGF cut-off value is between 30 and 90 μg/mL.

In an additional aspect, the present invention provides the method of treating RVO in a subject as disclosed herein, wherein the VEGF cut-off value is about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, or about 85 μg/mL.

In a further aspect, the present invention provides the method of treating RVO in a subject as disclosed herein, wherein the bl-CMT cut-off value is between 250-600 μm.

In an additional aspect, the present invention provides the method of treating RVO in a subject as disclosed herein, wherein the bl-CMT cut-off value is about 250 μm, about 300 μm, about 350 μm, about 400 μm, about 450 μm, about 500 μm, about 550 μm, or about 600 μm.

In a further aspect, the present invention provides the method of treating RVO in a subject as disclosed herein, wherein the level of VEGF is determined by an immunoassay, optionally an enzymatic immunoassay.

In an additional aspect, the present invention provides the method of treating RVO in a subject as disclosed herein, wherein the subject is in need of treatment of retinal vein occlusion (RVO).

In a further aspect, the present invention provides the method of treating RVO in a subject as disclosed herein, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

In an additional aspect, the present invention provides the method of treating RVO in a subject as disclosed herein, further comprising administering the anti-VEGF therapeutic agent or another anti-VEGF therapeutic agent intravitreally, suprachoroidally, and/or subretinally to the subject when the determined VEGF level is higher than the VEGF cut-off value and when the determined bl-CMT level is higher than the bl-CMT cut-off value.

In a further aspect, the present invention provides the method of treating RVO in a subject as disclosed herein, wherein the determined VEGF level is higher than 90 μg/mL, and the anti-VEGF therapeutic agent or another anti-VEGF therapeutic agent is administered intravitreally, suprachoroidally, and/or subretinally to the subject.

In an additional aspect, the present invention provides the method of treating RVO in a subject as disclosed herein, wherein the subject has not been treated with the anti-VEGF therapeutic agent or another anti-VEGF therapeutic agent prior to the determining step.

In a further aspect, the present invention provides the method of treating RVO in a subject as disclosed herein, wherein the subject has been treated with the anti-VEGF therapeutic agent or another anti-VEGF therapeutic agent prior to the determining step.

In an additional aspect, the present invention provides the method of treating RVO in a subject as disclosed herein, wherein the subject has not been treated with a steroid, optionally a corticosteroid, and further optionally a glucocorticosteroid, prior to the determining step.

In a further aspect, the present invention provides the method of treating RVO in a subject as disclosed herein, wherein the subject has been treated with a steroid, optionally a corticosteroid, and further optionally a glucocorticosteroid, prior to the determining step.

In an additional aspect, the present invention provides the method of treating RVO in a subject as disclosed herein, wherein the subject has not been treated with an anti-inflammatory or immunosuppressive agent, or in combination, to the subject, optionally wherein the anti-inflammatory or immunosuppressive agent is glucocorticosteroid, methotrexate, cyclosporin A, tacrolimus, azathioprine, or a biologic agent targeting a pro-inflammatory molecule such as interleukin-6, interleukin-8, tumor necrosis factor-alpha, etc.

In a further aspect, the present invention provides the method of treating RVO in a subject as disclosed herein, wherein the subject has been treated with an anti-inflammatory or immunosuppressive agent, or in combination, to the subject, optionally wherein the anti-inflammatory or immunosuppressive agent is glucocorticosteroid, methotrexate, cyclosporin A, tacrolimus, azathioprine, or a biologic agent targeting a pro-inflammatory molecule such as interleukin-6, interleukin-8, tumor necrosis factor-alpha, etc.

In an additional aspect, the present invention provides the method of treating RVO in a subject as disclosed herein, wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In a further aspect, the present invention provides the method of treating RVO in a subject as disclosed herein, wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In an additional aspect, the present invention provides the method of treating RVO in a subject as disclosed herein,

In a further aspect, the present invention provides the method of treating RVO in a subject as disclosed herein, wherein the subject is a human.

In an additional aspect, the present invention provides the method of treating RVO in a subject as disclosed herein, wherein the anti-VEGF therapeutic agent is formulated for intravitreal, suprachoroidal, and/or subretinal administration, optionally wherein the method further comprises admixing the anti-VEGF therapeutic agent and a pharmaceutically acceptable excipient or carrier.

In a further aspect, the present invention provides a use of an anti-VEGF therapeutic agent for the manufacture of a medicament for treating retinal vein occlusion (RVO) in a subject, wherein a level of VEGF in the aqueous humor of the subject is higher than a cut-off value, and the cut-off value is between 10 and 100 μg/mL.

In an additional aspect, the present invention provides the use of an anti-VEGF therapeutic agent for the manufacture of a medicament for treating RVO in a subject as disclosed herein, wherein the anti-VEGF therapeutic agent comprises:
an anti-VEGF antibody or antigen binding fragment thereof, optionally wherein the antigen binding fragment is Fab, Fab′, F(ab′)₂, or Fv;
   a VEGF receptor (VEGFR) fusion protein, optionally wherein the VEGFR fusion protein is a VEGFR-Fc immunoadhesin; or
   an anti-VEGF gene therapy agent, optionally wherein the anti-VEGF gene therapy agent comprises a nucleic acid encoding a VEGF antagonist, a nucleic acid encoding a protein that downregulates VEGF/VEGFR expression, and/or a nucleic acid targeting nucleic acids and/or proteins involved in VEGF/VEGFR expression, optionally wherein the anti-VEGF gene therapy agent comprises a viral or non-viral vector, optionally wherein the viral vector is an adeno-associated virus (AAV) vector.

In a further aspect, the present invention provides the use of an anti-VEGF therapeutic agent for the manufacture of a medicament for treating RVO in a subject as disclosed herein, wherein the anti-VEGF therapeutic agent is administered or prepared to be administered intravitreally, suprachoroidally, and/or subretinally.

In an additional aspect, the present invention provides the use of an anti-VEGF therapeutic agent for the manufacture of a medicament for treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In a further aspect, the present invention provides the use of an anti-VEGF therapeutic agent for the manufacture of a medicament for treating RVO in a subject as disclosed herein, wherein the subject has been or has not been treated with the anti-VEGF therapeutic agent or another anti-VEGF therapeutic agent.

In an additional aspect, the present invention provides the use of an anti-VEGF therapeutic agent for the manufacture of a medicament for treating RVO in a subject as disclosed herein, wherein the subject has been or has not been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In a further aspect, the present invention provides the use of an anti-VEGF therapeutic agent for the manufacture of a medicament for treating RVO in a subject as disclosed herein, wherein the subject has been or has not been treated with a steroid, optionally wherein the steroid is a corticosteroid, and optionally wherein the corticosteroid is a glucocorticosteroid.

In an additional aspect, the present invention provides the use of an anti-VEGF therapeutic agent for the manufacture of a medicament for treating RVO in a subject as disclosed herein, wherein the cut-off value is between 30 and 90 μg/mL.

In a further aspect, the present invention provides the use of an anti-VEGF therapeutic agent for the manufacture of a medicament for treating RVO in a subject as disclosed herein, wherein the cut-off value is about 35, about 40, about 45, about 50, about 55, about 60, about 65, about 70, about 75, about 80, or about 85 μg/mL.

In an additional aspect, the present invention provides the use of an anti-VEGF therapeutic agent for the manufacture of a medicament for treating RVO in a subject as disclosed herein, wherein the subject has an anatomical responsiveness to the anti-VEGF therapeutic agent as measured by change of central macular thickness (CMT) or central foveal thickness (CFT), wherein the CMT or CFT is defined as the vertical distance between the internal limiting membrane and the retinal pigment epithelium at the foveal center, or as measured by relative change of central macular thickness (CMT), wherein the relative change of CMT is defined by change of CMT divided by baseline central macular thickness (bl-CMT).

In a further aspect, the present invention provides the use of an anti-VEGF therapeutic agent for the manufacture of a medicament for treating RVO in a subject as disclosed herein, wherein the subject has a level of baseline central macular thickness (bl-CMT) higher than a bl-CMT cut-off value, wherein the bl-CMT cut-off value is between about 250 and about 600 μm.

In an additional aspect, the present invention provides a use of an anti-VEGF therapeutic agent for the manufacture of a kit for treating retinal vein occlusion (RVO), wherein the kit comprises: i) the anti-VEGF therapeutic agent and ii) instructions of administering the anti-VEGF therapeutic agent to a subject having a level of VEGF in the aqueous humor higher than a cut-off value, and wherein the cut-off value is between 10 and 100 μg/mL.

In a further aspect, the present invention provides the use of an anti-VEGF therapeutic agent for the manufacture of a kit for treating RVO as disclosed herein, wherein the kit further comprises instructions of not administering the anti-VEGF therapeutic agent to a subject having a level of VEGF in the aqueous humor no higher than the cut-off value, and/or instructions of administering an anti-inflammatory agent or an immunosuppressive agent, or in combination, to the subject having a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In an additional aspect, the present invention provides the use of an anti-VEGF therapeutic agent for the manufacture of a kit for treating RVO as disclosed herein, wherein the subject having a level of VEGF in the aqueous humor higher than the cut-off value has a level of bl-CMT higher than a bl-CMT cut-off value, and wherein the bl-CMT cut-off value is between about 250 and about 600 μm.

In a further aspect, the present invention provides a kit comprising i) an anti-VEGF therapeutic agent and ii) instructions of administering the anti-VEGF therapeutic agent to a subject having a level of VEGF in the aqueous humor higher than a cut-off value, wherein the cut-off value is between 10 and 100 μg/mL.

In a further aspect, the present invention provides the kit as disclosed herein, further comprising instructions of not administering the anti-VEGF therapeutic agent to a subject having a level of VEGF in the aqueous humor no higher than the cut-off value, and or instructions of administering an anti-inflammatory agent or an immunosuppressive agent, or in combination, to the subject having a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In an additional aspect, the present invention provides the kit as disclosed herein, wherein the subject having a level of VEGF in the aqueous humor higher than the cut-off value has a level of bl-CMT higher than a bl-CMT cut-off value, and wherein the bl-CMT cut-off value is between about 250 and about 600 μm.

In a further aspect, the present invention provides a kit comprising: i) a reagent for determining a level of VEGF in the aqueous humor of a subject, and ii) instructions comprising a cut-off value of VEGF level and optionally instructions of comparing the determined level of VEGF to the cut-off value, wherein the cut-off value is between 10 and 100 μg/mL.

In an additional aspect, the present invention provides the kit as disclosed herein further comprising a first standard sample having a level of VEGF that substantially equals the cut-off value.

In a further aspect, the present invention provides the kit as disclosed herein further comprising a second standard sample having a level of VEGF higher than the cut-off value.

In an additional aspect, the present invention provides the kit as disclosed herein further comprising a third standard sample having a level of VEGF lower than the cut-off value.

In a further aspect, the present invention provides the kit as disclosed herein, wherein the reagent is an anti-VEGF antibody or antigen binding fragment thereof, optionally wherein the antigen binding fragment is Fab, Fab′, F(ab′)₂, or Fv.

In an additional aspect, the present invention provides the kit as disclosed herein comprising an enzyme reactant, a magnetic separation reagent, a stabilizer, standards, one or more concentrated wash buffers, and/or substrate buffers.

In a further aspect, the present invention provides the kit as disclosed herein, wherein the enzyme reactant comprises a VEGF-specific antibody I labeled with an alkaline phosphatase, and wherein the labeling ratio of the VEGF antibody I to the alkaline phosphatase is 1:0.75.

In an additional aspect, the present invention provides the kit as disclosed herein, wherein the magnetic separation reagent contains 0.05 mass percent of magnetic particles, wherein the magnetic particles are coated with a VEGF-specific antibody II, wherein the magnetic reagent further comprises a blocking agent, and wherein the blocking agent is a mutant alkaline phosphatase.

In a further aspect, the present invention provides the kit as disclosed herein, wherein the VEGF-specific antibody I and/or the VEGF-specific antibody II is an anti-VEGF165/VEGFA antibody.

In an additional aspect, the present invention provides the kit as disclosed herein, wherein the stabilizer comprises one or more reagents selected from the group consisting of Tris, NaCl, Tween20, bovine serum albumin, mutant alkaline phosphatase, and ProClin 300.

In a further aspect, the present invention provides the kit as disclosed herein, wherein the wash buffer comprises Tris, NaCl, Tween-20, Triton-100, and ProClin 300.

In an additional aspect, the present invention provides a composition comprising i) isolated aqueous humor from a subject, and ii) a reagent that directly or indirectly interacts with VEGF in the isolated aqueous humor, wherein the isolated aqueous humor comprises a level of VEGF higher than a cut-off value, and the cut-off value is between 10 and 100 μg/mL.

In a further aspect, the present invention provides the composition as disclosed herein, wherein the reagent is an anti-VEGF antibody or antigen binding fragment thereof, optionally wherein the antigen binding fragment is Fab, Fab′, F(ab′)₂, or Fv.

In an additional aspect, the present invention provides the composition as disclosed herein, wherein the reagent comprises a VEGF-specific antibody I labeled with an alkaline phosphatase, and wherein the labeling ratio of the VEGF antibody I to the alkaline phosphatase is 1:0.75.

In a further aspect, the present invention provides the composition as disclosed herein, wherein the reagent comprises 0.05 mass percent of magnetic particles, wherein the magnetic particles are coated with a VEGF-specific antibody II, wherein the reagent further comprises a blocking agent, and wherein the blocking agent is a mutant alkaline phosphatase.

In an additional aspect, the present invention provides the composition as disclosed herein, wherein the VEGF-specific antibody I and/or the VEGF-specific antibody II is an anti-VEGF165/VEGFA antibody.

In a further aspect, the present invention provides an anti-VEGF therapeutic agent for use in treating retinal vein occlusion (RVO) in a subject, wherein a level of VEGF in the aqueous humor of the subject is higher than a cut-off value, and the cut-off value is between 10 and 100 μg/mL.

In an additional aspect, the present invention provides the anti-VEGF therapeutic agent for use in treating RVO in a subject as disclosed herein, comprising:
an anti-VEGF antibody or antigen binding fragment thereof, optionally wherein the antigen binding fragment is Fab, Fab′, F(ab′)₂, or Fv;
a VEGF receptor (VEGFR) fusion protein, optionally wherein the VEGFR fusion protein is a VEGFR-Fc immunoadhesin; or
an anti-VEGF gene therapy agent comprising a nucleic acid encoding a VEGF antagonist, a nucleic acid encoding a protein that downregulates VEGF/VEGFR expression, and/or a nucleic acid targeting nucleic acids and/or proteins involved in VEGF/VEGFR expression, optionally wherein the anti-VEGF gene therapy agent comprises a viral or non-viral vector, optionally wherein the viral vector is an adeno-associated virus (AAV) vector.

In a further aspect, the present invention provides the anti-VEGF therapeutic agent for use in treating RVO in a subject as disclosed herein, wherein the anti-VEGF therapeutic agent is administered or prepared to be administered intravitreally, suprachoroidally, and/or subretinally.

In an additional aspect, the present invention provides the anti-VEGF therapeutic agent for use in treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In a further aspect, the present invention provides the anti-VEGF therapeutic agent for use in treating RVO in a subject as disclosed herein, wherein the subject has been or has not been treated with the anti-VEGF therapeutic agent or another anti-VEGF therapeutic agent.

In an additional aspect, the present invention provides the anti-VEGF therapeutic agent for use in treating RVO in a subject as disclosed herein, wherein the subject has been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In a further aspect, the present invention provides the anti-VEGF therapeutic agent for use in treating RVO in a subject as disclosed herein, wherein the subject has not been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In an additional aspect, the present invention provides the anti-VEGF therapeutic agent for use in treating RVO in a subject as disclosed herein, wherein the cut-off value is between 30 and 90 μg/mL.

In a further aspect, the present invention provides the anti-VEGF therapeutic agent for use in treating RVO in a subject as disclosed herein, wherein the cut-off value is about 35, about 40, about 45, about 50, about 55, about 60, about 65, about 70, about 75, about 80, or about 85 μg/mL.

In an additional aspect, the present invention provides the anti-VEGF therapeutic agent for use in treating RVO in a subject as disclosed herein, wherein the subject has an anatomical response to the anti-VEGF therapeutic agent as measured by change of central macular thickness (CMT) or central foveal thickness (CFT), or as measured by relative change of central macular thickness (CMT), wherein the relative change of CMT is defined by change of CMT divided by baseline central macular thickness (bl-CMT).

In a further aspect, the present invention provides the anti-VEGF therapeutic agent for use in treating RVO in a subject as disclosed herein, wherein a level of baseline central macular thickness (bl-CMT) in the subject is higher than a bl-CMT cut-off value, and wherein the bl-CMT cut-off value is between about 250 and about 600 μm.

In an additional aspect, the present invention provides a method for predicting anatomical response to an anti-VEGF therapeutic agent treatment in a subject's eye, comprising determining a VEGF level in the aqueous humor collected from the subject, wherein a determined VEGF level higher than a cut-off value indicates that the eye has a good anatomical response to the anti-VEGF therapeutic agent treatment, wherein a determined VEGF level no higher than the cut-off value indicates that the eye has a poor anatomical response to the anti-VEGF therapeutic agent treatment, and wherein the cut-off value is between 10 and 100 μg/mL,

In a further aspect, the present invention provides the method for predicting anatomical response to an anti-VEGF therapeutic agent treatment in a subject's eye as disclosed herein, wherein the anatomical response to the anti-VEGF therapeutic agent is measured by change of central macular thickness (CMT) or central foveal thickness (CFT), wherein the CMT or CFT is defined as the vertical distance between the internal limiting membrane and the retinal pigment epithelium at the foveal center; or wherein the anatomical response to the anti-VEGF therapeutic agent is measured by relative change of central macular thickness (CMT) as defined by change of CMT divided by baseline central macular thickness (bl-CMT).

In an additional aspect, the present invention provides the method for predicting anatomical response to an anti-VEGF therapeutic agent treatment in a subject's eye as disclosed herein, wherein a determined VEGF level higher than the cut-off value predicts a change of CFT<−200 μm.

In a further aspect, the present invention provides the method for predicting anatomical response to an anti-VEGF therapeutic agent treatment in a subject's eye as disclosed herein, wherein a determined VEGF level lower than the cut-off value predicts a change of CFT>−200 μm.

In an additional aspect, the present invention provides the method for predicting anatomical response to an anti-VEGF therapeutic agent treatment in a subject's eye as disclosed herein, wherein the cut-off value is between 30 and 90 μg/mL.

In a further aspect, the present invention provides the method for predicting anatomical response to an anti-VEGF therapeutic agent treatment in a subject's eye as disclosed herein, wherein the cut-off value is about 35, about 40, about 45, about 50, about 55, about 60, about 65, about 70, about 75, about 80, or about 85 μg/mL.

In an additional aspect, the present invention provides the method for predicting anatomical response to an anti-VEGF therapeutic agent treatment in a subject's eye as disclosed herein, wherein a determined VEGF level higher than 90 μg/mL predicts the eye has a good anatomical response to the anti-VEGF therapeutic agent treatment.

In a further aspect, the present invention provides the method for predicting anatomical response to an anti-VEGF therapeutic agent treatment in a subject's eye as disclosed herein, wherein a determined VEGF level lower than 30 μg/mL predicts the eye has a poor anatomical response to the anti-VEGF therapeutic agent treatment.

In an additional aspect, the present invention provides the method for predicting anatomical response to an anti-VEGF therapeutic agent treatment in a subject's eye as disclosed herein, wherein the level of VEGF is determined by an immunoassay, optionally an enzymatic immunoassay.

In a further aspect, the present invention provides the method for predicting anatomical response to an anti-VEGF therapeutic agent treatment in a subject's eye as disclosed herein, wherein the subject is in need of treatment of retinal vein occlusion (RVO).

In an additional aspect, the present invention provides the method for predicting anatomical response to an anti-VEGF therapeutic agent treatment in a subject's eye as disclosed herein, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

In a further aspect, the present invention provides the method for predicting anatomical response to an anti-VEGF therapeutic agent treatment in a subject's eye as disclosed herein, further comprising administering the anti-VEGF therapeutic agent or another anti-VEGF therapeutic agent intravitreally, suprachoroidally, and/or subretinally to the subject when the determined VEGF level is higher than the cut-off value.

In an additional aspect, the present invention provides the method for predicting anatomical response to an anti-VEGF therapeutic agent treatment in a subject's eye as disclosed herein, wherein the determined VEGF level is higher than 90 μg/mL, and the anti-VEGF therapeutic agent or another anti-VEGF therapeutic agent is administered intravitreally, suprachoroidally, and/or subretinally to the subject.

In a further aspect, the present invention provides the method for predicting anatomical response to an anti-VEGF therapeutic agent treatment in a subject's eye as disclosed herein further comprising administering a therapy other than the anti-VEGF therapeutic agent to the subject when the determined VEGF level is lower than the cut-off value.

In an additional aspect, the present invention provides the method for predicting anatomical response to an anti-VEGF therapeutic agent treatment in a subject's eye as disclosed herein, wherein the determined VEGF level is lower than 30 μg/mL, and the anti-VEGF therapeutic agent or another anti-VEGF therapeutic agent is not administered intravitreally, suprachoroidally, and/or subretinally to the subject.

In a further aspect, the present invention provides the method for predicting anatomical response to an anti-VEGF therapeutic agent treatment in a subject's eye as disclosed herein, wherein the subject has not been treated with the anti-VEGF therapeutic agent or another anti-VEGF therapeutic agent prior to the determining step.

In an additional aspect, the present invention provides the method for predicting anatomical response to an anti-VEGF therapeutic agent treatment in a subject's eye as disclosed herein, wherein the subject has been treated with the anti-VEGF therapeutic agent or another anti-VEGF therapeutic agent prior to the determining step.

In a further aspect, the present invention provides the method for predicting anatomical response to an anti-VEGF therapeutic agent treatment in a subject's eye as disclosed herein, wherein the subject has not been treated with a steroid, optionally a corticosteroid, and further optionally a glucocorticosteroid, prior to the determining step.

In an additional aspect, the present invention provides the method for predicting anatomical response to an anti-VEGF therapeutic agent treatment in a subject's eye as disclosed herein, wherein the subject has been treated with a steroid, optionally a corticosteroid, and further optionally a glucocorticosteroid, prior to the determining step.

In a further aspect, the present invention provides the method for predicting anatomical response to an anti-VEGF therapeutic agent treatment in a subject's eye as disclosed herein, wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In an additional aspect, the present invention provides the method for predicting anatomical response to an anti-VEGF therapeutic agent treatment in a subject's eye as disclosed herein, wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In a further aspect, the present invention provides the method for predicting anatomical response to an anti-VEGF therapeutic agent treatment in a subject's eye as disclosed herein, wherein the subject is a human.

In an additional aspect, the present invention provides the method for predicting anatomical response to an anti-VEGF therapeutic agent treatment in a subject's eye as disclosed herein, wherein the anti-VEGF therapeutic agent is formulated for intravitreal, suprachoroidal, and/or subretinal administration, optionally wherein the method further comprises admixing the anti-VEGF therapeutic agent and a pharmaceutically acceptable excipient or carrier.

In a further aspect, the present invention provides the method for predicting anatomical response to an anti-VEGF therapeutic agent treatment in a subject's eye as disclosed herein, further comprising determining a bl-CMT level in the subject, wherein a determined bl-CMT level higher than a bl-CMT cut-off value indicates that the eye has a good anatomical response to the anti-VEGF therapeutic agent treatment, wherein a determined bl-CMT level no higher than the bl-CMT cut-off value indicates that the eye has a poor anatomical response to the anti-VEGF therapeutic agent treatment, and wherein the bl-CMT cut-off value is between about 250 and about 600 μm.

In an additional aspect, the present invention provides a method for predicting anatomical response to an anti-VEGF therapeutic agent treatment in an eye of a subject, comprising determining the VEGF level in the aqueous humor collected from the subject and the bl-CMT level in the subject, wherein the determined VEGF level higher than the VEGF cut-off value and the determined bl-CMT level higher than the bl-CMT cut-off value indicate that the eye has a good anatomical response to the anti-VEGF therapeutic agent treatment; and wherein the determined VEGF level no higher than the VEGF cut-off value or the determined bl-CMT level no higher than the bl-CMT cut-off value indicate that the eye has a poor anatomical response to the anti-VEGF therapeutic agent treatment.

In a further aspect, the present invention provides the method for predicting anatomical response to an anti-VEGF therapeutic agent treatment in an eye of a subject as disclosed herein, wherein the VEGF cut-off value is between 10 and 100 μg/mL.

In an additional aspect, the present invention provides the method for predicting anatomical response to an anti-VEGF therapeutic agent treatment in an eye of a subject as disclosed herein, wherein the bl-CMT cut-off value is between about 250 and about 600 μm.

In a further aspect, the present invention provides the method for predicting anatomical response to an anti-VEGF therapeutic agent treatment in an eye of a subject as disclosed herein, wherein the anatomical response to the anti-VEGF therapeutic agent is measured by change of CMT which is defined as the vertical distance between the internal limiting membrane and the retinal pigment epithelium at the foveal center.

In an additional aspect, the present invention provides the method for predicting anatomical response to an anti-VEGF therapeutic agent treatment in an eye of a subject as disclosed herein, wherein the anatomical response to the anti-VEGF therapeutic agent is measured by relative change of CMT, wherein the relative change of CMT is defined by change of CMT divided by bl-CMT.

In a further aspect, the present invention provides the method for predicting anatomical response to an anti-VEGF therapeutic agent treatment in an eye of a subject as disclosed herein, wherein the determined VEGF level higher than a VEGF cut-off value of between 10 and 100 μg/mL and the determined bl-CMT level higher than a bl-CMT cut-off value of between about 250 and about 600 μm indicate that the eye has a good anatomical response to the anti-VEGF therapeutic agent treatment.

In an additional aspect, the present invention provides the method for predicting anatomical response to an anti-VEGF therapeutic agent treatment in an eye of a subject as disclosed herein, wherein the determined VEGF level higher than the VEGF cut-off value and the determined bl-CMT level higher than the bl-CMT cut-off value predict a change of CFT<−200 μm.

In a further aspect, the present invention provides the method for predicting anatomical response to an anti-VEGF therapeutic agent treatment in an eye of a subject as disclosed herein, wherein the determined VEGF level lower than the VEGF cut-off value or the determined bl-CMT level lower than the bl-CMT cut-off value predicts a change of CFT>−200 μm.

In a further aspect, the present invention provides the method for predicting anatomical response to an anti-VEGF therapeutic agent treatment in an eye of a subject as disclosed herein, wherein the VEGF cut-off value is between 30 and 90 μg/mL.

In an additional aspect, the present invention provides the method for predicting anatomical response to an anti-VEGF therapeutic agent treatment in an eye of a subject as disclosed herein, wherein the VEGF cut-off value is about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, or about 85 μg/mL.

In a further aspect, the present invention provides the method for predicting anatomical response to an anti-VEGF therapeutic agent treatment in an eye of a subject as disclosed herein, wherein the bl-CMT cut-off value is between 250-600 μm.

In an additional aspect, the present invention provides the method for predicting anatomical response to an anti-VEGF therapeutic agent treatment in an eye of a subject as disclosed herein, wherein the bl-CMT cut-off value is about 250 μm, about 300 μm, about 350 μm, about 400 μm, about 450 μm, about 500 μm, about 550 μm, or about 600 μm.

In an additional aspect, the present invention provides the method for predicting anatomical response to an anti-VEGF therapeutic agent treatment in an eye of a subject as disclosed herein, wherein the level of VEGF is determined by an immunoassay, optionally an enzymatic immunoassay.

In a further aspect, the present invention provides the method for predicting anatomical response to an anti-VEGF therapeutic agent treatment in an eye of a subject as disclosed herein, wherein the subject is in need of treatment of retinal vein occlusion (RVO).

In an additional aspect, the present invention provides the method for predicting anatomical response to an anti-VEGF therapeutic agent treatment in an eye of a subject as disclosed herein, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

In an additional aspect, the present invention provides the method for predicting anatomical response to an anti-VEGF therapeutic agent treatment in an eye of a subject as disclosed herein, further comprising administering the anti-VEGF therapeutic agent or another anti-VEGF therapeutic agent intravitreally, suprachoroidally, and/or subretinally to the subject when the determined VEGF level is higher than the VEGF cut-off value and when the determined bl-CMT level is higher than the bl-CMT cut-off value.

In a further aspect, the present invention provides the method for predicting anatomical response to an anti-VEGF therapeutic agent treatment in an eye of a subject as disclosed herein, wherein the determined VEGF level is higher than 90 μg/mL, and the anti-VEGF therapeutic agent or another anti-VEGF therapeutic agent is administered intravitreally, suprachoroidally, and/or subretinally to the subject.

In an additional aspect, the present invention provides the method for predicting anatomical response to an anti-VEGF therapeutic agent treatment in an eye of a subject as disclosed herein, further comprising administering a therapy other than the anti-VEGF therapeutic agent to the subject when the determined VEGF level is lower than the VEGF cut-off value.

In a further aspect, the present invention provides the method for predicting anatomical response to an anti-VEGF therapeutic agent treatment in an eye of a subject as disclosed herein, wherein the determined VEGF level is lower than 30 μg/mL, and the anti-VEGF therapeutic agent or another anti-VEGF therapeutic agent is not administered intravitreally, suprachoroidally, and/or subretinally to the subject.

In an additional aspect, the present invention provides the method for predicting anatomical response to an anti-VEGF therapeutic agent treatment in an eye of a subject as disclosed herein, wherein the therapy other than the anti-VEGF therapeutic agent is a steroid, optionally a corticosteroid, and further optionally a glucocorticosteroid.

In a further aspect, the present invention provides the method for predicting anatomical response to an anti-VEGF therapeutic agent treatment in an eye of a subject as disclosed herein, wherein the subject has been or has not been treated with the anti-VEGF therapeutic agent or another anti-VEGF therapeutic agent prior to the determining step.

In an additional aspect, the present invention provides the method for predicting anatomical response to an anti-VEGF therapeutic agent treatment in an eye of a subject as disclosed herein, wherein the subject has been or has not been treated with a steroid, optionally a corticosteroid, and further optionally a glucocorticosteroid, prior to the determining step.

In a further aspect, the present invention provides the method for predicting anatomical response to an anti-VEGF therapeutic agent treatment in an eye of a subject as disclosed herein, wherein the subject has been or has not been treated with an anti-inflammatory agent or immunosuppressive agent, or in combination, to the subject, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In an additional aspect, the present invention provides the method for predicting anatomical response to an anti-VEGF therapeutic agent treatment in an eye of a subject as disclosed herein, wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In a further aspect, the present invention provides the method for predicting anatomical response to an anti-VEGF therapeutic agent treatment in an eye of a subject as disclosed herein, wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In an additional aspect, the present invention provides the method for predicting anatomical response to an anti-VEGF therapeutic agent treatment in an eye of a subject as disclosed herein, wherein the subject is a human.

In an additional aspect, the present invention provides the method for predicting anatomical response to an anti-VEGF therapeutic agent treatment in an eye of a subject as disclosed herein,

In a further aspect, the present invention provides the method for predicting anatomical response to an anti-VEGF therapeutic agent treatment in an eye of a subject as disclosed herein, wherein the anti-VEGF therapeutic agent is formulated for intravitreal, suprachoroidal, and/or subretinal administration, optionally wherein the method further comprises admixing the anti-VEGF therapeutic agent and a pharmaceutically acceptable excipient or carrier.

### BRIEF DESCRIPTION OF THE DRAWINGS

The drawings illustrate certain features and advantages of this disclosure. These embodiments are not intended to limit the scope of the appended claims in any manner.

FIG. 1. depicts changes of central foveal thickness (CFT, defined as the vertical distance between the internal limiting membrane and the retinal pigment epithelium at the foveal center) or maximum paravoveal retinal thickness (MRT) when fovea was not involved to 4 weeks after the intravitreal injection as compared to baseline plotted against baseline aqueous VEGF level.

FIG. 2. Comparisons of baseline aqueous humor cytokines concentrations and clinical parameters between the two subgroups. Figures A-H: comparisons of aqueous interleukin (IL)-6, IL-8, interferon-gamma-inducible protein-10 (IP-10), IL-10, monocyte chemoattractant protein-1 (MCP-1), vascular endothelial growth factor (VEGF) detected by magnetic-particle-based chemiluminescence enzyme immunoassay kit (MECLEIA), baseline central macular thickness (bl-CMT) and best corrected visual acuity (bl-BCVA) (logarithm of the minimum angle of resolution (logMAR) scale) between non-treated and treated groups.

FIG. 3. Receiver Operating Characteristic (ROC) curves for identification of cut-off values and assessments of regression models. FIGS. 3A and 3B: Cut-off values of aqueous interleukin (IL)-8, and vascular endothelial growth factor (VEGF) detected by magnetic-particle-based chemiluminescence enzyme immunoassay kit (VEGF-MECLEIA) for prediction of ATR (as measured by central macular thickness change (ACMT)/baseline central macular thickness (bl-CMT)). FIG. 3C: Area under curve (AUC) for multivariate logistic regression models of VEGF+bl-CMT. FIGS. 3D and 3E: AUC for multivariate logistic regression models of treatment response with VEGF and bl-CMT.

FIG. 4. Representative cases in the good response (GR) and poor response (PR) groups. FIGS. 4A and 4B were baseline fundus photograph and optical coherence tomography (OCT) scan of a representative patient in the GR group and non-treated group. Before intravitreal ranibizumab injection (IVR), baseline central macular thickness (bl-CMT) and baseline central retinal thickness (bl-CRT) were 404 and 357 μm respectively; aqueous VEGF detected by the magnetic-particle-based chemiluminescence enzyme immunoassay kit (VEGF-MPCLEIA) was 129.8 pg/ml respectively. FIGS. 4C and 4D were fundus photograph and OCT scan of this case 5 weeks after IVR. CMT and CRT decreased to 148 and 195 μm respectively with complete resolution of retinal edema. FIGS. 4E and 4F were baseline fundus photograph and OCT scan of a representative case in the PR group and treated group. bl-CMT and bl-CRT were 714 and 405 μm respectively. FIGS. 4G and 4H were fundus photograph and OCT scan of the same patient 5 weeks after the anti-VEGF injection, CMT and CRT were 730 and 402 μm, respectively.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

In some embodiments, provided herein is a use of aqueous VEGF level as a companion biomarker for anti-VEGF treatments in RVO, and methods and compositions associated with the use. In some embodiments, a level of VEGF in the aqueous humor of a subject can be used to predict responsiveness of the subject to one or more anti-VEGF therapeutic agents. In some embodiments, a level of VEGF in the aqueous humor higher than a VEGF cut-off value or range between about 10 μg/mL and about 100 μg/mL can be used as a companion diagnostic to predict anatomical treatment response to anti-VEGF in patients. In some embodiments, an anti-VEGF therapeutic agent herein is an agent that targets a signaling pathway mediated by VEGF, e.g., through VEGFR. In some embodiments, the anti-VEGF therapeutic agent targets VEGF. In some embodiments, the anti-VEGF therapeutic agent targets VEGFR.

In some embodiments, provided herein is a use of aqueous VEGF level in combination with bl-CMT level as companion biomarker(s) for anti-VEGF treatments in RVO, and methods and compositions associated with the use. In some embodiments, a level of VEGF in the aqueous humor of a subject in combination of a level of bl-CMT in the subject can be used to predict responsiveness of the subject to one or more anti-VEGF therapeutic agents. In some embodiments, a level of VEGF in the aqueous humor higher than a VEGF cut-off value or range between about 10 μg/mL and about 100 μg/mL and a level of bl-CMT in the subject higher than a bl-CMT cut-off value or range between about 250 and about 600 μm can be used as a companion diagnostic to predict anatomical treatment response to anti-VEGF in patients. In some embodiments, an anti-VEGF therapeutic agent herein is an agent that targets a signaling pathway mediated by VEGF, e.g., through VEGFR. In some embodiments, the anti-VEGF therapeutic agent targets VEGF. In some embodiments, the anti-VEGF therapeutic agent targets VEGFR.

In any of the embodiments herein, the anti-VEGF therapeutic agent can comprise an anti-VEGF antibody or antigen binding fragment thereof, optionally wherein the antigen binding fragment is Fab, Fab′, F(ab′)2, or Fv.

In any of the embodiments herein, the anti-VEGF therapeutic agent can comprise a VEGF receptor (VEGFR) fusion protein, optionally wherein the VEGFR fusion protein is a VEGFR-Fc immunoadhesin.

In any of the embodiments herein, the anti-VEGF therapeutic agent can comprise an anti-VEGF gene therapy agent. In any of the embodiments herein, the anti-VEGF therapeutic agent can comprise a nucleic acid encoding a VEGF antagonist. In any of the embodiments herein, the anti-VEGF therapeutic agent can comprise a nucleic acid encoding a VEGFR antagonist. In any of the embodiments herein, the anti-VEGF therapeutic agent can comprise a nucleic acid encoding a protein or a nucleic acid that down-regulates VEGF and/or VEGFR expression. In any of the embodiments herein, the anti-VEGF therapeutic agent can comprise a nucleic acid that down-regulates VEGF and/or VEGFR expression.

In any of the embodiments herein, the anti-VEGF therapeutic agent can comprise a viral or non-viral vector, optionally wherein the viral vector is an adeno-associated virus (AAV) vector.

In any of the embodiments herein, the anti-VEGF therapeutic agent can comprise nucleic acid(s) (either DNA or RNA) that target nucleic acids or proteins involved in VEGF and/or VEGFR expression. In any of the embodiments herein, the anti-VEGF therapeutic agent can comprise VEGF antisense oligodeoxynucleotides and/or VEGFR antisense oligodeoxynucleotides.

In any of the embodiments herein, the anti-VEGF therapeutic agent can be administered or prepared to be administered intravitreally, suprachoroidally, and/or subretinally. In any of the embodiments herein, the subject can suffer from macular edema secondary to retinal vein occlusion (ME-RVO), optionally wherein the retinal vein occlusion is central retinal vein occlusion (CRVO) and/or branch retinal vein occlusion (BRVO).

In any of the embodiments herein, the subject can have been treated with the anti-VEGF therapeutic agent or another anti-VEGF therapeutic agent. In any of the embodiments herein, the subject may have not been treated with the anti-VEGF therapeutic agent or another anti-VEGF therapeutic agent. In any of the embodiments herein, the subject may have been treated with a steroid, optionally wherein the steroid is a corticosteroid, and optionally wherein the corticosteroid is a glucocorticosteroid. In any of the embodiments herein, the subject may have not been treated with a steroid, optionally wherein the steroid is a corticosteroid, and optionally wherein the corticosteroid is a glucocorticosteroid. In any of the embodiments herein, the subject may have been treated with an anti-inflammatory agent or an immunosuppressive agent, or in combination, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha. In any of the embodiments herein, the subject may have not been treated with an anti-inflammatory agent or an immunosuppressive agent, or in combination, optionally wherein the anti-inflammatory agent or immunosuppressive agent is selected from the group consisting of glucocorticosteroid,

In any of the embodiments herein, the VEGF cut-off value may be between 30 and 90 μg/mL. In any of the embodiments herein, the VEGF cut-off value may be about 35, about 40, about 45, about 50, about 55, about 60, about 65, about 70, about 75, about 80, or about 85 μg/mL.

In any of the embodiments herein, the subject can have an anatomical responsiveness to the anti-VEGF therapeutic agent or called “anatomical treatment response (ATR)” which is measured by change of central foveal thickness (CFT, defined as the vertical distance between the internal limiting membrane and the retinal pigment epithelium at the foveal center), or change of central macular thickness (CMT, defined as the vertical distance between the internal limiting membrane and the retinal pigment epithelium at the foveal center), relative change of CMT (defined as change of CMT divided by baseline central macular thickness (bl-CMT), i.e. ΔCMT/bl-CMT), change of mean macular thickness (MMT, defined as average retinal thickness from all 9 regions of ETDRS map), or change of maximum paravoveal retinal thickness (MRT), or other measurements derived from anyone of the above measurements. In any of the embodiments herein, the subject can have an anatomical responsiveness to the anti-VEGF therapeutic agent as measured by ΔCFT/CFT. In some embodiments, an anatomical responsiveness to the anti-VEGF therapeutic agent or ATR is measured by change of CFT or CMT. In some embodiments, an anatomical responsiveness to the anti-VEGF therapeutic agent or ATR is measured by relative change of CMT. A relative CMT change of ≤−25% after the intravitreal injection (ΔCMT/bl-CMT) is categorized as a good anatomical response or a good ATR. A relative CMT change of >-25% after the intravitreal injection (ΔCMT/bl-CMT) is categorized as a poor anatomical response or a poor ATR.

In some embodiments, provided herein is a use of an anti-VEGF therapeutic agent for the manufacture of a kit for treating retinal vein occlusion (RVO), wherein the kit comprises: i) the anti-VEGF therapeutic agent and ii) instructions of administering the anti-VEGF therapeutic agent to a subject having a level of VEGF in the aqueous humor higher than a VEGF cut-off value, and wherein the VEGF cut-off value is between 10 and 100 μg/mL. In some embodiments, the kit further comprises instructions of not administering the anti-VEGF therapeutic agent to a subject having a level of VEGF in the aqueous humor no higher than the VEGF cut-off value. In any of the embodiments herein, the kit can comprise instructions of administering a steroid to the subject having a level of VEGF in the aqueous humor no higher than the VEGF cut-off value, optionally wherein the steroid is a corticosteroid, and optionally wherein the corticosteroid is a glucocorticosteroid. In any of the embodiments herein, the kit can comprise instructions of administering an anti-inflammatory agent or an immunosuppressive agent, or in combination, to the subject having a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, provided herein is a use of an anti-VEGF therapeutic agent for the manufacture of a kit for treating retinal vein occlusion (RVO), wherein the kit comprises: i) the anti-VEGF therapeutic agent and ii) instructions of administering the anti-VEGF therapeutic agent to a subject having a level of VEGF in the aqueous humor higher than a VEGF cut-off value between 10 and 100 μg/mL, and having a level of bl-CMT higher than a bl-CMT cut-off value between about 250 and about 600 μm. In some embodiments, the kit further comprises instructions of not administering the anti-VEGF therapeutic agent to a subject having a level of VEGF in the aqueous humor no higher than the VEGF cut-off value or a level of bl-CMT no higher than the bl-CMT cut-off value. In any of the embodiments herein, the kit can comprise instructions of administering a steroid to the subject having a level of VEGF in the aqueous humor no higher than the VEGF cut-off value or having a level of bl-CMT no higher than the bl-CMT cut-off value, optionally wherein the steroid is a corticosteroid, and optionally wherein the corticosteroid is a glucocorticosteroid. In any of the embodiments herein, the kit can comprise instructions of administering an anti-inflammatory agent or an immunosuppressive agent, or in combination, to the subject having a level of VEGF in the aqueous humor higher than the cut-off value or having a level of bl-CMT no higher than the bl-CMT cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, provided herein is a kit comprising i) an anti-VEGF therapeutic agent and ii) instructions of administering the anti-VEGF therapeutic agent to a subject having a level of VEGF in the aqueous humor higher than a VEGF cut-off value, wherein the VEGF cut-off value is between 10 and 100 μg/mL. In any of the embodiments herein, the kit can comprise instructions of not administering the anti-VEGF therapeutic agent to a subject having a level of VEGF in the aqueous humor no higher than the VEGF cut-off value. In any of the embodiments herein, the kit can comprise instructions of administering a steroid to the subject having a level of VEGF in the aqueous humor no higher than the VEGF cut-off value, optionally wherein the steroid is a corticosteroid, and optionally wherein the corticosteroid is a glucocorticosteroid.

In some embodiments, provided herein is a kit comprising: i) a reagent for determining a level of VEGF in the aqueous humor of a subject, and ii) instructions comprising a VEGF cut-off value of VEGF level and optionally instructions of comparing the determined level of VEGF to the VEGF cut-off value, wherein the VEGF cut-off value is between 10 and 100 μg/mL.

In any of the embodiments herein, the kit can comprise a first standard sample having a level of VEGF that substantially equals the VEGF cut-off value. In any of the embodiments herein, the kit can comprise a second standard sample having a level of VEGF higher than the VEGF cut-off value. In any of the embodiments herein, the kit can comprise a third standard sample having a level of VEGF lower than the VEGF cut-off value.

In any of the embodiments herein, the reagent can be an anti-VEGF antibody or antigen binding fragment thereof, optionally wherein the antigen binding fragment is Fab, Fab′, F(ab′)2, or Fv.

In any of the embodiments herein, the kit can comprise an enzyme reactant, a magnetic separation reagent, a stabilizer, standards, one or more concentrated wash buffers, and/or substrate buffers. In any of the embodiments herein, the enzyme reactant can comprise a VEGF-specific antibody I labeled with an alkaline phosphatase, and wherein the labeling ratio of the VEGF antibody I to the alkaline phosphatase is 1:0.75. In any of the embodiments herein, the magnetic separation reagent may contain 0.05 mass percent of magnetic particles, wherein magnetic particles are coated with a VEGF-specific antibody II, wherein the magnetic reagent further comprises a blocking agent, and wherein the blocking agent is a mutant alkaline phosphatase. In any of the embodiments herein, the VEGF-specific antibody I and/or the VEGF-specific antibody II can be an anti-VEGF165/VEGFA antibody. In any of the embodiments herein, the stabilizer can comprise one or more of Tris, NaCl, Tween20, bovine serum albumin, mutant alkaline phosphatase, and ProClin 300. In any of the embodiments herein, the wash buffer can comprise Tris, NaCl, Tween-20, Triton-100, and ProClin 300.

In some embodiments, provided herein is a composition comprising i) isolated aqueous humor from a subject, and ii) a reagent that directly or indirectly interacts with VEGF in the isolated aqueous humor, wherein the isolated aqueous humor comprises a level of VEGF higher than a VEGF cut-off value, and the VEGF cut-off value is between 10 and 100 μg/mL.

In some embodiments, provided herein is a composition comprising i) isolated aqueous humor from a subject, and ii) a reagent that directly or indirectly interacts with VEGF in the isolated aqueous humor, wherein the isolated aqueous humor comprise a level of VEGF no higher than a VEGF cut-off value, and the VEGF cut-off value is between 10 and 100 μg/mL.

In any of the embodiments herein, the reagent can be an anti-VEGF antibody or antigen binding fragment thereof, optionally wherein the antigen binding fragment is Fab, Fab′, F(ab′)2, or Fv. In any of the embodiments herein, the reagent may comprise a VEGF-specific antibody I labeled with an alkaline phosphatase, and wherein the labeling ratio of the VEGF antibody I to the alkaline phosphatase is 1:0.75. In any of the embodiments herein, the reagent may comprise 0.05 mass percent of magnetic particles, wherein magnetic particles are coated with a VEGF-specific antibody II, wherein the reagent further comprises a blocking agent, and wherein the blocking agent is a mutant alkaline phosphatase. In any of the embodiments herein, the VEGF-specific antibody I and/or the VEGF-specific antibody II can be an anti-VEGF165/VEGFA antibody.

In some embodiments disclosed herein is an anti-VEGF therapeutic agent for use in treating retinal vein occlusion (RVO) in a subject, wherein a level of VEGF in the aqueous humor of the subject is higher than a VEGF cut-off value, and the VEGF cut-off value is between 10 and 100 μg/mL.

In any of the embodiments herein, the anti-VEGF therapeutic agent may comprise an anti-VEGF antibody or antigen binding fragment thereof, optionally wherein the antigen binding fragment is Fab, Fab′, F(ab′)2, or Fv. In any of the embodiments herein, the anti-VEGF therapeutic agent may comprisea VEGF receptor (VEGFR) fusion protein, optionally wherein the VEGFR fusion protein is a VEGFR-Fc immunoadhesin.

In any of the embodiments herein, the anti-VEGF therapeutic agent may comprise an anti-VEGF gene therapy agent comprising a nucleic acid encoding a VEGF antagonist, optionally wherein the anti-VEGF gene therapy agent comprises a viral or non-viral vector, optionally wherein the viral vector is aan adeno-associated virus (AAV) vector. In any of the embodiments herein, the anti-VEGF therapeutic agent can comprise a nucleic acid encoding a VEGFR antagonist. In any of the embodiments herein, the anti-VEGF therapeutic agent can comprisea nucleic acid encoding a protein or a nucleic acid that down-regulates VEGF and/or VEGFR expression. In any of the embodiments herein, the anti-VEGF therapeutic agent can comprisea nucleic acid that down-regulates VEGF and/or VEGFR expression. In any of the embodiments herein, the anti-VEGF therapeutic agent can comprise nucleic acid(s) (either DNA or RNA) that target nucleic acids or proteins involved in VEGF and/or VEGFR expression. In any of the embodiments herein, the anti-VEGF therapeutic agent can comprise VEGF antisense oligodeoxynucleotides and/or VEGFR antisense oligodeoxynucleotides. In any of the embodiments herein, the anti-VEGF therapeutic agent may be administered or prepared to be administered intravitreally, suprachoroidally, and/or subretinally.

In any of the embodiments herein, the subject may suffer from macular edema secondary to retinal vein occlusion (ME-RVO), optionally wherein the retinal vein occlusion is central retinal vein occlusion (CRVO) and/or branch retinal vein occlusion (BRVO).

In any of the embodiments herein, the subject may have been treated with the anti-VEGF therapeutic agent or another anti-VEGF therapeutic agent.

In any of the embodiments herein, the subject may have not been treated with the anti-VEGF therapeutic agent or another anti-VEGF therapeutic agent.

In any of the embodiments herein, the subject may have been treated with a steroid, optionally wherein the steroid is a corticosteroid, and optionally wherein the corticosteroid is a glucocorticosteroid.

In any of the embodiments herein, the subject may have not been treated with a steroid, optionally wherein the steroid is a corticosteroid, and optionally wherein the corticosteroid is a glucocorticosteroid.

In any of the embodiments herein, the VEGF cut-off value can include a range, that is, the VEGF cut-off may be a VEGF cut-off range. In any of the embodiments herein, the VEGF cut-off can be any value or range between 10 and 100 μg/mL. In any of the embodiments herein, the VEGF cut-off can be any value or range between 30 and 90 μg/mL. In any of the embodiments herein, the VEGF cut-off can be about 35, about 40, about 45, about 50, about 55, about 60, about 65, about 70, about 75, about 80, or about 85 μg/mL.

In any of the embodiments herein, the subject can have an anatomical response to the anti-VEGF therapeutic agent as measured by change of CFT.

In some embodiments, disclosed herein is a method for determining anatomical response to an anti-VEGF therapeutic agent treatment in a subject's eye, comprising determining a VEGF level in the aqueous humor collected from the subject, wherein a determined VEGF level higher than a VEGF cut-off indicates that the eye has a good anatomical response to the anti-VEGF therapeutic agent treatment, wherein a determined VEGF level no higher than the VEGF cut-off value indicates that the eye has a poor anatomical response to the anti-VEGF therapeutic agent treatment, and wherein the VEGF cut-off is between 10 and 100 μg/mL.

In any of the embodiments herein, the anatomical response to the anti-VEGF therapeutic agent can be measured by change of CFT. In any of the embodiments herein, a determined VEGF level higher than the VEGF cut-off can be associated with and/or used to predict a change of CFT<−200 μm. In any of the embodiments herein, a determined VEGF level lower than the VEGF cut-off can be associated with and/or used to predict a change of CFT>−200 μm.

In any of the embodiments herein, the VEGF cut-off can be between 30 and 90 μg/mL. In any of the embodiments herein, the VEGF cut-off can be about 35, about 40, about 45, about 50, about 55, about 60, about 65, about 70, about 75, about 80, or about 85 μg/mL.

In any of the embodiments herein, a determined VEGF level higher than 90 μg/mL can be associated with and/or used to predict the eye has a good anatomical response to the anti-VEGF therapeutic agent treatment.

In any of the embodiments herein, a determined VEGF level lower than 30 μg/mL can be associated with and/or used to predict the eye has a poor anatomical response to the anti-VEGF therapeutic agent treatment.

In any of the embodiments herein, the level of VEGF can be determined by an immunoassay, optionally an enzymatic immunoassay.

In any of the embodiments herein, the subject may be in need of treatment of retinal vein occlusion (RVO). In any of the embodiments herein, the subject may be in need of treatment of macular edema secondary to retinal vein occlusion (ME-RVO), optionally wherein the retinal vein occlusion is central retinal vein occlusion (CRVO) and/or branch retinal vein occlusion (BRVO).

In any of the embodiments herein, the method can further comprise administering the anti-VEGF therapeutic agent or another anti-VEGF therapeutic agent intravitreally, suprachoroidally, and/or subretinally to the subject when the determined VEGF level is higher than the VEGF cut-off value.

In any of the embodiments herein, the determined VEGF level can be higher than 90 μg/mL, and the anti-VEGF therapeutic agent or another anti-VEGF therapeutic agent is administered intravitreally, suprachoroidally, and/or subretinally to the subject. In some embodiments, a steroid, optionally a corticosteroid, and further optionally a glucocorticosteroid, is not administered to the subject.

In any of the embodiments herein, the method can further comprise administering a therapy other than the anti-VEGF therapeutic agent to the subject when the determined VEGF level is lower than the VEGF cut-off value.

In any of the embodiments herein, the determined VEGF level can be lower than 30 μg/mL, and the anti-VEGF therapeutic agent or another anti-VEGF therapeutic agent is not administered intravitreally, suprachoroidally, and/or subretinally to the subject.

In any of the embodiments herein, the therapy other than the anti-VEGF therapeutic agent can be a steroid, optionally a corticosteroid, and further optionally a glucocorticosteroid.

In some embodiments, the present invention provides a method of treating retinal vein occlusion (RVO) in a subject, comprising administrating an effective amount of an anti-VEGF therapeutic agent to the subject, wherein a level of VEGF in the aqueous humor of the subject is higher than a VEGF cut-off value, and the VEGF cut-off value is between 10 and 100 μg/mL.

In some embodiments, the present invention provides the method of treating RVO in a subject as disclosed herein, wherein the anti-VEGF therapeutic agent comprises:
an anti-VEGF antibody or antigen binding fragment thereof, optionally wherein the antigen binding fragment is Fab, Fab′, F(ab′)2, or Fv;
a VEGF receptor (VEGFR) fusion protein, optionally wherein the VEGFR fusion protein is a VEGFR-Fc immunoadhesin; or
an anti-VEGF gene therapy agent, optionally wherein the anti-VEGF gene therapy agent comprises a nucleic acid encoding a VEGF antagonist, a nucleic acid encoding a protein that downregulates VEGF/VEGFR expression, and/or a nucleic acid targeting nucleic acids and/or proteins involved in VEGF/VEGFR expression, optionally wherein the anti-VEGF gene therapy agent comprises a viral or non-viral vector, optionally wherein the viral vector is an adeno-associated virus (AAV) vector.

In some embodiments, the present invention provides the method of treating RVO in a subject as disclosed herein, wherein the anti-VEGF therapeutic agent is administered or prepared to be administered intravitreally, suprachoroidally, and/or subretinally.

In some embodiments, the present invention provides the method of treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the method of treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the method of treating RVO in a subject as disclosed herein, wherein the subject has been treated with the anti-VEGF therapeutic agent or another anti-VEGF therapeutic agent.

In some embodiments, the present invention provides the method of treating RVO in a subject as disclosed herein, wherein the subject has not been treated with the anti-VEGF therapeutic agent or another anti-VEGF therapeutic agent.

In some embodiments, the present invention provides the method of treating RVO in a subject as disclosed herein, wherein the subject has been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the method of treating RVO in a subject as disclosed herein, wherein the subject has not been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the method of treating RVO in a subject as disclosed herein, wherein the subject has been treated with a steroid, optionally wherein the steroid is a corticosteroid, and optionally wherein the corticosteroid is a glucocorticosteroid.

In some embodiments, the present invention provides the method of treating RVO in a subject as disclosed herein, wherein the subject has not been treated with a steroid, optionally wherein the steroid is a corticosteroid, and optionally wherein the corticosteroid is a glucocorticosteroid.

In some embodiments, the present invention provides the method of treating RVO in a subject as disclosed herein, wherein the VEGF cut-off value is between 30 and 90 μg/mL.

In some embodiments, the present invention provides the method of treating RVO in a subject as disclosed herein, wherein the VEGF cut-off value is about 35, about 40, about 45, about 50, about 55, about 60, about 65, about 70, about 75, about 80, or about 85 μg/mL.

In some embodiments, the present invention provides the method of treating RVO in a subject as disclosed herein, wherein the subject has an anatomical responsiveness to the anti-VEGF therapeutic agent as measured by change of central macular thickness (CMT) or central foveal thickness (CFT), wherein the CMT or CFT is defined as the vertical distance between the internal limiting membrane and the retinal pigment epithelium at the foveal center and is also described as central foveal thickness (CFT) in some circumstances.

In some embodiments, the present invention provides the method of treating RVO in a subject as disclosed herein, wherein the subject has an anatomical responsiveness to the anti-VEGF therapeutic agent as measured by relative change of central macular thickness (CMT), wherein the relative change of CMT is defined by change of CMT divided by baseline central macular thickness (bl-CMT).

In some embodiments, the present invention provides the method of treating RVO in a subject as disclosed herein, wherein the subject has a level of baseline central macular thickness (bl-CMT) higher than a bl-CMT cut-off value, wherein the bl-CMT cut-off value is between about 250 and about 600 μm.

In some embodiments, the present invention provides a method of treating retinal vein occlusion (RVO) in a subject as disclosed herein, comprising:
(a) determining the level of VEGF in the aqueous humor collected from the subject and the bl-CMT level in the subject, wherein the determined VEGF level is higher than the VEGF cut-off value and the determined bl-CMT level is higher than the bl-CMT cut-off value,
(b) administrating an effective amount of an anti-VEGF therapeutic agent to the subject.

In some embodiments, the present invention provides the method of treating RVO in a subject as disclosed herein, wherein the VEGF cut-off value is between 10 and 100 μg/mL.

In some embodiments, the present invention provides the method of treating RVO in a subject as disclosed herein, wherein the bl-CMT cut-off value is between about 250 and about 600 μm.

In some embodiments, the present invention provides the method of treating RVO in a subject as disclosed herein,

In some embodiments, the present invention provides the method of treating RVO in a subject as disclosed herein, wherein the anatomical response to the anti-VEGF therapeutic agent is measured by change of CMT which is defined as the vertical distance between the internal limiting membrane and the retinal pigment epithelium at the foveal center.

In some embodiments, the present invention provides the method of treating RVO in a subject as disclosed herein, wherein the anatomical response to the anti-VEGF therapeutic agent is measured by relative change of CMT, wherein the relative change of CMT is defined by change of CMT divided by bl-CMT.

In some embodiments, the present invention provides the method of treating RVO in a subject as disclosed herein, wherein the determined VEGF level higher than a VEGF cut-off value of between 10 and 100 μg/mL and the determined bl-CMT level higher than a bl-CMT cut-off value of between about 250 and about 600 μm indicate that the eye has a good anatomical response to the anti-VEGF therapeutic agent treatment.

In some embodiments, the present invention provides the method of treating RVO in a subject as disclosed herein, wherein the determined VEGF level higher than the VEGF cut-off value and the determined bl-CMT level higher than the bl-CMT cut-off value predict a change of CFT<−200 μm.

In some embodiments, the present invention provides the method of treating RVO in a subject as disclosed herein, wherein the VEGF cut-off value is between 30 and 90 μg/mL.

In some embodiments, the present invention provides the method of treating RVO in a subject as disclosed herein, wherein the VEGF cut-off value is about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, or about 85 μg/mL.

In some embodiments, the present invention provides the method of treating RVO in a subject as disclosed herein, wherein the bl-CMT cut-off value is between 250-600 μm.

In some embodiments, the present invention provides the method of treating RVO in a subject as disclosed herein, wherein the bl-CMT cut-off value is about 250 μm, about 300 μm, about 350 μm, about 400 μm, about 450 μm, about 500 μm, about 550 μm, or about 600 μm.

In some embodiments, the present invention provides the method of treating RVO in a subject as disclosed herein, wherein the level of VEGF is determined by an immunoassay, optionally an enzymatic immunoassay.

In some embodiments, the present invention provides the method of treating RVO in a subject as disclosed herein, wherein the subject is in need of treatment of retinal vein occlusion (RVO).

In some embodiments, the present invention provides the method of treating RVO in a subject as disclosed herein, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the method of treating RVO in a subject as disclosed herein, further comprising administering the anti-VEGF therapeutic agent or another anti-VEGF therapeutic agent intravitreally, suprachoroidally, and/or subretinally to the subject when the determined VEGF level is higher than the VEGF cut-off value and when the determined bl-CMT level is higher than the bl-CMT cut-off value.

In some embodiments, the present invention provides the method of treating RVO in a subject as disclosed herein, wherein the determined VEGF level is higher than 90 μg/mL, and the anti-VEGF therapeutic agent or another anti-VEGF therapeutic agent is administered intravitreally, suprachoroidally, and/or subretinally to the subject.

In some embodiments, the present invention provides the method of treating RVO in a subject as disclosed herein, wherein the subject has not been treated with the anti-VEGF therapeutic agent or another anti-VEGF therapeutic agent prior to the determining step.

In some embodiments, the present invention provides the method of treating RVO in a subject as disclosed herein, wherein the subject has been treated with the anti-VEGF therapeutic agent or another anti-VEGF therapeutic agent prior to the determining step.

In some embodiments, the present invention provides the method of treating RVO in a subject as disclosed herein, wherein the subject has not been treated with a steroid, optionally a corticosteroid, and further optionally a glucocorticosteroid, prior to the determining step.

In some embodiments, the present invention provides the method of treating RVO in a subject as disclosed herein, wherein the subject has been treated with a steroid, optionally a corticosteroid, and further optionally a glucocorticosteroid, prior to the determining step.

In some embodiments, the present invention provides the method of treating RVO in a subject as disclosed herein, wherein the subject has not been treated with an anti-inflammatory or immunosuppressive agent, or in combination, to the subject, optionally wherein the anti-inflammatory or immunosuppressive agent is glucocorticosteroid, methotrexate, cyclosporin A, tacrolimus, azathioprine, or a biologic agent targeting a pro-inflammatory molecule such as interleukin-6, interleukin-8, tumor necrosis factor-alpha, etc.

In some embodiments, the present invention provides the method of treating RVO in a subject as disclosed herein, wherein the subject has been treated with an anti-inflammatory or immunosuppressive agent, or in combination, to the subject, optionally wherein the anti-inflammatory or immunosuppressive agent is glucocorticosteroid, methotrexate, cyclosporin A, tacrolimus, azathioprine, or a biologic agent targeting a pro-inflammatory molecule such as interleukin-6, interleukin-8, tumor necrosis factor-alpha, etc.

In some embodiments, the present invention provides the method of treating RVO in a subject as disclosed herein, wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In some embodiments, the present invention provides the method of treating RVO in a subject as disclosed herein, wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In some embodiments, the present invention provides the method of treating RVO in a subject as disclosed herein,

In some embodiments, the present invention provides the method of treating RVO in a subject as disclosed herein, wherein the subject is a human.

In some embodiments, the present invention provides the method of treating RVO in a subject as disclosed herein, wherein the anti-VEGF therapeutic agent is formulated for intravitreal, suprachoroidal, and/or subretinal administration, optionally wherein the method further comprises admixing the anti-VEGF therapeutic agent and a pharmaceutically acceptable excipient or carrier.

In some embodiments, the present invention provides a use of an anti-VEGF therapeutic agent for the manufacture of a medicament for treating retinal vein occlusion (RVO) in a subject, wherein a level of VEGF in the aqueous humor of the subject is higher than a cut-off value, and the cut-off value is between 10 and 100 μg/mL.

In some embodiments, the present invention provides the use of an anti-VEGF therapeutic agent for the manufacture of a medicament for treating RVO in a subject as disclosed herein, wherein the anti-VEGF therapeutic agent comprises:
an anti-VEGF antibody or antigen binding fragment thereof, optionally wherein the antigen binding fragment is Fab, Fab′, F(ab′)2, or Fv;
   a VEGF receptor (VEGFR) fusion protein, optionally wherein the VEGFR fusion protein is a VEGFR-Fc immunoadhesin; or
   an anti-VEGF gene therapy agent, optionally wherein the anti-VEGF gene therapy agent comprises a nucleic acid encoding a VEGF antagonist, a nucleic acid encoding a protein that downregulates VEGF/VEGFR expression, and/or a nucleic acid targeting nucleic acids and/or proteins involved in VEGF/VEGFR expression, optionally wherein the anti-VEGF gene therapy agent comprises a viral or non-viral vector, optionally wherein the viral vector is an adeno-associated virus (AAV) vector.

In some embodiments, the present invention provides the use of an anti-VEGF therapeutic agent for the manufacture of a medicament for treating RVO in a subject as disclosed herein, wherein the anti-VEGF therapeutic agent is administered or prepared to be administered intravitreally, suprachoroidally, and/or subretinally.

In some embodiments, the present invention provides the use of an anti-VEGF therapeutic agent for the manufacture of a medicament for treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the use of an anti-VEGF therapeutic agent for the manufacture of a medicament for treating RVO in a subject as disclosed herein, wherein the subject has been or has not been treated with the anti-VEGF therapeutic agent or another anti-VEGF therapeutic agent.

In some embodiments, the present invention provides the use of an anti-VEGF therapeutic agent for the manufacture of a medicament for treating RVO in a subject as disclosed herein, wherein the subject has been or has not been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the use of an anti-VEGF therapeutic agent for the manufacture of a medicament for treating RVO in a subject as disclosed herein, wherein the subject has been or has not been treated with a steroid, optionally wherein the steroid is a corticosteroid, and optionally wherein the corticosteroid is a glucocorticosteroid.

In some embodiments, the present invention provides the use of an anti-VEGF therapeutic agent for the manufacture of a medicament for treating RVO in a subject as disclosed herein, wherein the cut-off value is between 30 and 90 μg/mL.

In some embodiments, the present invention provides the use of an anti-VEGF therapeutic agent for the manufacture of a medicament for treating RVO in a subject as disclosed herein, wherein the cut-off value is about 35, about 40, about 45, about 50, about 55, about 60, about 65, about 70, about 75, about 80, or about 85 μg/mL.

In some embodiments, the present invention provides the use of an anti-VEGF therapeutic agent for the manufacture of a medicament for treating RVO in a subject as disclosed herein, wherein the subject has an anatomical responsiveness to the anti-VEGF therapeutic agent as measured by change of central macular thickness (CMT) or central foveal thickness (CFT), wherein the CMT or CFT is defined as the vertical distance between the internal limiting membrane and the retinal pigment epithelium at the foveal center, or as measured by relative change of central macular thickness (CMT), wherein the relative change of CMT is defined by change of CMT divided by baseline central macular thickness (bl-CMT).

In some embodiments, the present invention provides the use of an anti-VEGF therapeutic agent for the manufacture of a medicament for treating RVO in a subject as disclosed herein, wherein the subject has a level of baseline central macular thickness (bl-CMT) higher than a bl-CMT cut-off value, wherein the bl-CMT cut-off value is between about 250 and about 600 μm.

In some embodiments, the present invention provides a use of an anti-VEGF therapeutic agent for the manufacture of a kit for treating retinal vein occlusion (RVO), wherein the kit comprises: i) the anti-VEGF therapeutic agent and ii) instructions of administering the anti-VEGF therapeutic agent to a subject having a level of VEGF in the aqueous humor higher than a cut-off value, and wherein the cut-off value is between 10 and 100 μg/mL.

In some embodiments, the present invention provides the use of an anti-VEGF therapeutic agent for the manufacture of a kit for treating RVO as disclosed herein, wherein the kit further comprises instructions of not administering the anti-VEGF therapeutic agent to a subject having a level of VEGF in the aqueous humor no higher than the cut-off value, and/or instructions of administering an anti-inflammatory agent or an immunosuppressive agent, or in combination, to the subject having a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the use of an anti-VEGF therapeutic agent for the manufacture of a kit for treating RVO as disclosed herein, wherein the subject having a level of VEGF in the aqueous humor higher than the cut-off value has a level of bl-CMT higher than a bl-CMT cut-off value, and wherein the bl-CMT cut-off value is between about 250 and about 600 μm.

In some embodiments, the present invention provides a kit comprising i) an anti-VEGF therapeutic agent and ii) instructions of administering the anti-VEGF therapeutic agent to a subject having a level of VEGF in the aqueous humor higher than a cut-off value, wherein the cut-off value is between 10 and 100 μg/mL.

In some embodiments, the present invention provides the kit as disclosed herein, further comprising instructions of not administering the anti-VEGF therapeutic agent to a subject having a level of VEGF in the aqueous humor no higher than the cut-off value, and or instructions of administering an anti-inflammatory agent or an immunosuppressive agent, or in combination, to the subject having a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the kit as disclosed herein, wherein the subject having a level of VEGF in the aqueous humor higher than the cut-off value has a level of bl-CMT higher than a bl-CMT cut-off value, and wherein the bl-CMT cut-off value is between about 250 and about 600 μm.

In some embodiments, the present invention provides a kit comprising: i) a reagent for determining a level of VEGF in the aqueous humor of a subject, and ii) instructions comprising a cut-off value of VEGF level and optionally instructions of comparing the determined level of VEGF to the cut-off value, wherein the cut-off value is between 10 and 100 μg/mL.

In some embodiments, the present invention provides the kit as disclosed herein further comprising a first standard sample having a level of VEGF that substantially equals the cut-off value.

In some embodiments, the present invention provides the kit as disclosed herein further comprising a second standard sample having a level of VEGF higher than the cut-off value.

In some embodiments, the present invention provides the kit as disclosed herein further comprising a third standard sample having a level of VEGF lower than the cut-off value.

In some embodiments, the present invention provides the kit as disclosed herein, wherein the reagent is an anti-VEGF antibody or antigen binding fragment thereof, optionally wherein the antigen binding fragment is Fab, Fab′, F(ab′)₂, or Fv.

In some embodiments, the present invention provides the kit as disclosed herein comprising an enzyme reactant, a magnetic separation reagent, a stabilizer, standards, one or more concentrated wash buffers, and/or substrate buffers.

In some embodiments, the present invention provides the kit as disclosed herein, wherein the enzyme reactant comprises a VEGF-specific antibody I labeled with an alkaline phosphatase, and wherein the labeling ratio of the VEGF antibody I to the alkaline phosphatase is 1:0.75.

In some embodiments, the present invention provides the kit as disclosed herein, wherein the magnetic separation reagent contains 0.05 mass percent of magnetic particles, wherein the magnetic particles are coated with a VEGF-specific antibody II, wherein the magnetic reagent further comprises a blocking agent, and wherein the blocking agent is a mutant alkaline phosphatase.

In some embodiments, the present invention provides the kit as disclosed herein, wherein the VEGF-specific antibody I and/or the VEGF-specific antibody II is an anti-VEGF165/VEGFA antibody.

In some embodiments, the present invention provides the kit as disclosed herein, wherein the stabilizer comprises one or more reagents selected from the group consisting of Tris, NaCl, Tween20, bovine serum albumin, mutant alkaline phosphatase, and ProClin 300.

In some embodiments, the present invention provides the kit as disclosed herein, wherein the wash buffer comprises Tris, NaCl, Tween-20, Triton-100, and ProClin 300.

In some embodiments, the present invention provides a composition comprising i) isolated aqueous humor from a subject, and ii) a reagent that directly or indirectly interacts with VEGF in the isolated aqueous humor, wherein the isolated aqueous humor comprises a level of VEGF higher than a cut-off value, and the cut-off value is between 10 and 100 μg/mL.

In some embodiments, the present invention provides the composition as disclosed herein, wherein the reagent is an anti-VEGF antibody or antigen binding fragment thereof, optionally wherein the antigen binding fragment is Fab, Fab′, F(ab′)₂, or Fv.

In some embodiments, the present invention provides the composition as disclosed herein, wherein the reagent comprises a VEGF-specific antibody I labeled with an alkaline phosphatase, and wherein the labeling ratio of the VEGF antibody I to the alkaline phosphatase is 1:0.75.

In some embodiments, the present invention provides the composition as disclosed herein, wherein the reagent comprises 0.05 mass percent of magnetic particles, wherein the magnetic particles are coated with a VEGF-specific antibody II, wherein the reagent further comprises a blocking agent, and wherein the blocking agent is a mutant alkaline phosphatase.

In some embodiments, the present invention provides the composition as disclosed herein, wherein the VEGF-specific antibody I and/or the VEGF-specific antibody II is an anti-VEGF165/VEGFA antibody.

In some embodiments, the present invention provides an anti-VEGF therapeutic agent for use in treating retinal vein occlusion (RVO) in a subject, wherein a level of VEGF in the aqueous humor of the subject is higher than a cut-off value, and the cut-off value is between 10 and 100 μg/mL.

In some embodiments, the present invention provides the anti-VEGF therapeutic agent for use in treating RVO in a subject as disclosed herein, comprising:
an anti-VEGF antibody or antigen binding fragment thereof, optionally wherein the antigen binding fragment is Fab, Fab′, F(ab′)₂, or Fv;
a VEGF receptor (VEGFR) fusion protein, optionally wherein the VEGFR fusion protein is a VEGFR-Fc immunoadhesin; or
an anti-VEGF gene therapy agent comprising a nucleic acid encoding a VEGF antagonist, a nucleic acid encoding a protein that downregulates VEGF/VEGFR expression, and/or a nucleic acid targeting nucleic acids and/or proteins involved in VEGF/VEGFR expression, optionally wherein the anti-VEGF gene therapy agent comprises a viral or non-viral vector, optionally wherein the viral vector is an adeno-associated virus (AAV) vector.

In some embodiments, the present invention provides the anti-VEGF therapeutic agent for use in treating RVO in a subject as disclosed herein, wherein the anti-VEGF therapeutic agent is administered or prepared to be administered intravitreally, suprachoroidally, and/or subretinally.

In some embodiments, the present invention provides the anti-VEGF therapeutic agent for use in treating RVO in a subject as disclosed herein, wherein the subject suffers from macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the anti-VEGF therapeutic agent for use in treating RVO in a subject as disclosed herein, wherein the subject has been or has not been treated with the anti-VEGF therapeutic agent or another anti-VEGF therapeutic agent.

In some embodiments, the present invention provides the anti-VEGF therapeutic agent for use in treating RVO in a subject as disclosed herein, wherein the subject has been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the anti-VEGF therapeutic agent for use in treating RVO in a subject as disclosed herein, wherein the subject has not been treated with an anti-inflammatory agent or an immunosuppressive agent, or in combination, wherein the subject has a level of VEGF in the aqueous humor higher than the cut-off value, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the anti-VEGF therapeutic agent for use in treating RVO in a subject as disclosed herein, wherein the cut-off value is between 30 and 90 μg/mL.

In some embodiments, the present invention provides the anti-VEGF therapeutic agent for use in treating RVO in a subject as disclosed herein, wherein the cut-off value is about 35, about 40, about 45, about 50, about 55, about 60, about 65, about 70, about 75, about 80, or about 85 μg/mL.

In some embodiments, the present invention provides the anti-VEGF therapeutic agent for use in treating RVO in a subject as disclosed herein, wherein the subject has an anatomical response to the anti-VEGF therapeutic agent as measured by change of central macular thickness (CMT) or central foveal thickness (CFT), or as measured by relative change of central macular thickness (CMT), wherein the relative change of CMT is defined by change of CMT divided by baseline central macular thickness (bl-CMT).

In some embodiments, the present invention provides the anti-VEGF therapeutic agent for use in treating RVO in a subject as disclosed herein, wherein a level of baseline central macular thickness (bl-CMT) in the subject is higher than a bl-CMT cut-off value, and wherein the bl-CMT cut-off value is between about 250 and about 600 μm.

In some embodiments, the present invention provides a method of predicting anatomical response to an anti-VEGF therapeutic agent treatment in a subject's eye, comprising evaluating or determining a VEGF level in the aqueous humor collected from the subject, wherein a determined VEGF level higher than a cut-off value indicates that the eye has a good anatomical response to the anti-VEGF therapeutic agent treatment, wherein a determined VEGF level no higher than the cut-off value indicates that the eye has a poor anatomical response to the anti-VEGF therapeutic agent treatment, and wherein the cut-off value is between 10 and 100 μg/mL,

In some embodiments, the present invention provides the method of predicting anatomical response to an anti-VEGF therapeutic agent treatment in a subject's eye as disclosed herein, wherein the anatomical response to the anti-VEGF therapeutic agent is measured by change of central macular thickness (CMT) or central foveal thickness (CFT), wherein the CMT or CFT is defined as the vertical distance between the internal limiting membrane and the retinal pigment epithelium at the foveal center; or wherein the anatomical response to the anti-VEGF therapeutic agent is measured by relative change of central macular thickness (CMT) as defined by change of CMT divided by baseline central macular thickness (bl-CMT).

In some embodiments, the present invention provides the method of predicting anatomical response to an anti-VEGF therapeutic agent treatment in a subject's eye as disclosed herein, wherein a determined VEGF level higher than the cut-off value predicts a change of CFT<−200 μm.

In some embodiments, the present invention provides the method of predicting anatomical response to an anti-VEGF therapeutic agent treatment in a subject's eye as disclosed herein, wherein a determined VEGF level lower than the cut-off value predicts a change of CFT>−200 μm.

In some embodiments, the present invention provides the method of predicting anatomical response to an anti-VEGF therapeutic agent treatment in a subject's eye as disclosed herein, wherein the cut-off value is between 30 and 90 μg/mL.

In some embodiments, the present invention provides the method of predicting anatomical response to an anti-VEGF therapeutic agent treatment in a subject's eye as disclosed herein, wherein the cut-off value is about 35, about 40, about 45, about 50, about 55, about 60, about 65, about 70, about 75, about 80, or about 85 μg/mL.

In some embodiments, the present invention provides the method of predicting anatomical response to an anti-VEGF therapeutic agent treatment in a subject's eye as disclosed herein, wherein a determined VEGF level higher than 90 μg/mL predicts the eye has a good anatomical response to the anti-VEGF therapeutic agent treatment.

In some embodiments, the present invention provides the method of predicting anatomical response to an anti-VEGF therapeutic agent treatment in a subject's eye as disclosed herein, wherein a determined VEGF level lower than 30 μg/mL predicts the eye has a poor anatomical response to the anti-VEGF therapeutic agent treatment.

In some embodiments, the present invention provides the method of predicting anatomical response to an anti-VEGF therapeutic agent treatment in a subject's eye as disclosed herein, wherein the level of VEGF is determined by an immunoassay, optionally an enzymatic immunoassay.

In some embodiments, the present invention provides the method of predicting anatomical response to an anti-VEGF therapeutic agent treatment in a subject's eye as disclosed herein, wherein the subject is in need of treatment of retinal vein occlusion (RVO).

In some embodiments, the present invention provides the method of predicting anatomical response to an anti-VEGF therapeutic agent treatment in a subject's eye as disclosed herein, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the method of predicting anatomical response to an anti-VEGF therapeutic agent treatment in a subject's eye as disclosed herein, further comprising administering the anti-VEGF therapeutic agent or another anti-VEGF therapeutic agent intravitreally, suprachoroidally, and/or subretinally to the subject when the determined VEGF level is higher than the cut-off value.

In some embodiments, the present invention provides the method of predicting anatomical response to an anti-VEGF therapeutic agent treatment in a subject's eye as disclosed herein, wherein the determined VEGF level is higher than 90 μg/mL, and the anti-VEGF therapeutic agent or another anti-VEGF therapeutic agent is administered intravitreally, suprachoroidally, and/or subretinally to the subject.

In some embodiments, the present invention provides the method of predicting anatomical response to an anti-VEGF therapeutic agent treatment in a subject's eye as disclosed herein further comprising administering a therapy other than the anti-VEGF therapeutic agent to the subject when the determined VEGF level is lower than the cut-off value.

In some embodiments, the present invention provides the method of predicting anatomical response to an anti-VEGF therapeutic agent treatment in a subject's eye as disclosed herein, wherein the determined VEGF level is lower than 30 μg/mL, and the anti-VEGF therapeutic agent or another anti-VEGF therapeutic agent is not administered intravitreally, suprachoroidally, and/or subretinally to the subject.

In some embodiments, the present invention provides the method of predicting anatomical response to an anti-VEGF therapeutic agent treatment in a subject's eye as disclosed herein, wherein the subject has not been treated with the anti-VEGF therapeutic agent or another anti-VEGF therapeutic agent prior to the determining step.

In some embodiments, the present invention provides the method of predicting anatomical response to an anti-VEGF therapeutic agent treatment in a subject's eye as disclosed herein, wherein the subject has been treated with the anti-VEGF therapeutic agent or another anti-VEGF therapeutic agent prior to the determining step.

In some embodiments, the present invention provides the method of predicting anatomical response to an anti-VEGF therapeutic agent treatment in a subject's eye as disclosed herein, wherein the subject has not been treated with a steroid, optionally a corticosteroid, and further optionally a glucocorticosteroid, prior to the determining step.

In some embodiments, the present invention provides the method of predicting anatomical response to an anti-VEGF therapeutic agent treatment in a subject's eye as disclosed herein, wherein the subject has been treated with a steroid, optionally a corticosteroid, and further optionally a glucocorticosteroid, prior to the determining step.

In some embodiments, the present invention provides the method of predicting anatomical response to an anti-VEGF therapeutic agent treatment in a subject's eye as disclosed herein, wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In some embodiments, the present invention provides the method of predicting anatomical response to an anti-VEGF therapeutic agent treatment in a subject's eye as disclosed herein, wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In some embodiments, the present invention provides the method of predicting anatomical response to an anti-VEGF therapeutic agent treatment in a subject's eye as disclosed herein, wherein the subject is a human.

In some embodiments, the present invention provides the method of predicting anatomical response to an anti-VEGF therapeutic agent treatment in a subject's eye as disclosed herein, wherein the anti-VEGF therapeutic agent is formulated for intravitreal, suprachoroidal, and/or subretinal administration, optionally wherein the method further comprises admixing the anti-VEGF therapeutic agent and a pharmaceutically acceptable excipient or carrier.

In some embodiments, the present invention provides the method of predicting anatomical response to an anti-VEGF therapeutic agent treatment in a subject's eye as disclosed herein, further comprising evaluating or determining a bl-CMT level in the subject, wherein a determined bl-CMT level higher than a bl-CMT cut-off value indicates that the eye has a good anatomical response to the anti-VEGF therapeutic agent treatment, wherein a determined bl-CMT level no higher than the bl-CMT cut-off value indicates that the eye has a poor anatomical response to the anti-VEGF therapeutic agent treatment, and wherein the bl-CMT cut-off value is between about 250 and about 600 μm.

In some embodiments, the present invention provides a method of predicting anatomical response to an anti-VEGF therapeutic agent treatment in an eye of a subject, comprising evaluating or determining the VEGF level in the aqueous humor collected from the subject and the bl-CMT level in the subject, wherein the determined VEGF level higher than the VEGF cut-off value and the determined bl-CMT level higher than the bl-CMT cut-off value indicate that the eye has a good anatomical response to the anti-VEGF therapeutic agent treatment; and wherein the determined VEGF level no higher than the VEGF cut-off value or the determined bl-CMT level no higher than the bl-CMT cut-off value indicate that the eye has a poor anatomical response to the anti-VEGF therapeutic agent treatment.

In some embodiments, the present invention provides the method of predicting anatomical response to an anti-VEGF therapeutic agent treatment in an eye of a subject as disclosed herein, wherein the VEGF cut-off value is between 10 and 100 μg/mL.

In some embodiments, the present invention provides the method of predicting anatomical response to an anti-VEGF therapeutic agent treatment in an eye of a subject as disclosed herein, wherein the bl-CMT cut-off value is between about 250 and about 600 μm.

In some embodiments, the present invention provides the method of predicting anatomical response to an anti-VEGF therapeutic agent treatment in an eye of a subject as disclosed herein, wherein the anatomical response to the anti-VEGF therapeutic agent is measured by change of CMT which is defined as the vertical distance between the internal limiting membrane and the retinal pigment epithelium at the foveal center.

In some embodiments, the present invention provides the method of predicting anatomical response to an anti-VEGF therapeutic agent treatment in an eye of a subject as disclosed herein, wherein the anatomical response to the anti-VEGF therapeutic agent is measured by relative change of CMT, wherein the relative change of CMT is defined by change of CMT divided by bl-CMT.

In some embodiments, the present invention provides the method of predicting anatomical response to an anti-VEGF therapeutic agent treatment in an eye of a subject as disclosed herein, wherein the determined VEGF level higher than a VEGF cut-off value of between 10 and 100 μg/mL and the determined bl-CMT level higher than a bl-CMT cut-off value of between about 250 and about 600 μm indicate that the eye has a good anatomical response to the anti-VEGF therapeutic agent treatment.

In some embodiments, the present invention provides the method of predicting anatomical response to an anti-VEGF therapeutic agent treatment in an eye of a subject as disclosed herein, wherein the determined VEGF level higher than the VEGF cut-off value and the determined bl-CMT level higher than the bl-CMT cut-off value predict a change of CFT<−200 μm.

In some embodiments, the present invention provides the method of predicting anatomical response to an anti-VEGF therapeutic agent treatment in an eye of a subject as disclosed herein, wherein the determined VEGF level lower than the VEGF cut-off value or the determined bl-CMT level lower than the bl-CMT cut-off value predicts a change of CFT>−200 μm.

In some embodiments, the present invention provides the method of predicting anatomical response to an anti-VEGF therapeutic agent treatment in an eye of a subject as disclosed herein, wherein the VEGF cut-off value is between 30 and 90 μg/mL.

In some embodiments, the present invention provides the method of predicting anatomical response to an anti-VEGF therapeutic agent treatment in an eye of a subject as disclosed herein, wherein the VEGF cut-off value is about 35 μg/mL, about 40 μg/mL, about 45 μg/mL, about 50 μg/mL, about 55 μg/mL, about 60 μg/mL, about 65 μg/mL, about 70 μg/mL, about 75 μg/mL, about 80 μg/mL, or about 85 μg/mL.

In some embodiments, the present invention provides the method of predicting anatomical response to an anti-VEGF therapeutic agent treatment in an eye of a subject as disclosed herein, wherein the bl-CMT cut-off value is between 250-600 μm.

In some embodiments, the present invention provides the method of predicting anatomical response to an anti-VEGF therapeutic agent treatment in an eye of a subject as disclosed herein, wherein the bl-CMT cut-off value is about 250 μm, about 300 μm, about 350 μm, about 400 μm, about 450 μm, about 500 μm, about 550 μm, or about 600 μm.

In some embodiments, the present invention provides the method of predicting anatomical response to an anti-VEGF therapeutic agent treatment in an eye of a subject as disclosed herein, wherein the level of VEGF is determined by an immunoassay, optionally an enzymatic immunoassay.

In some embodiments, the present invention provides the method of predicting anatomical response to an anti-VEGF therapeutic agent treatment in an eye of a subject as disclosed herein, wherein the subject is in need of treatment of retinal vein occlusion (RVO).

In some embodiments, the present invention provides the method of predicting anatomical response to an anti-VEGF therapeutic agent treatment in an eye of a subject as disclosed herein, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

In some embodiments, the present invention provides the method of predicting anatomical response to an anti-VEGF therapeutic agent treatment in an eye of a subject as disclosed herein, further comprising administering the anti-VEGF therapeutic agent or another anti-VEGF therapeutic agent intravitreally, suprachoroidally, and/or subretinally to the subject when the determined VEGF level is higher than the VEGF cut-off value and when the determined bl-CMT level is higher than the bl-CMT cut-off value.

In some embodiments, the present invention provides the method of predicting anatomical response to an anti-VEGF therapeutic agent treatment in an eye of a subject as disclosed herein, wherein the determined VEGF level is higher than 90 μg/mL, and the anti-VEGF therapeutic agent or another anti-VEGF therapeutic agent is administered intravitreally, suprachoroidally, and/or subretinally to the subject.

In some embodiments, the present invention provides the method of predicting anatomical response to an anti-VEGF therapeutic agent treatment in an eye of a subject as disclosed herein, further comprising administering a therapy other than the anti-VEGF therapeutic agent to the subject when the determined VEGF level is lower than the VEGF cut-off value.

In some embodiments, the present invention provides the method of predicting anatomical response to an anti-VEGF therapeutic agent treatment in an eye of a subject as disclosed herein, wherein the determined VEGF level is lower than 30 μg/mL, and the anti-VEGF therapeutic agent or another anti-VEGF therapeutic agent is not administered intravitreally, suprachoroidally, and/or subretinally to the subject.

In some embodiments, the present invention provides the method of predicting anatomical response to an anti-VEGF therapeutic agent treatment in an eye of a subject as disclosed herein, wherein the therapy other than the anti-VEGF therapeutic agent is a steroid, optionally a corticosteroid, and further optionally a glucocorticosteroid.

In some embodiments, the present invention provides the method of predicting anatomical response to an anti-VEGF therapeutic agent treatment in an eye of a subject as disclosed herein, wherein the subject has been or has not been treated with the anti-VEGF therapeutic agent or another anti-VEGF therapeutic agent prior to the determining step.

In some embodiments, the present invention provides the method of predicting anatomical response to an anti-VEGF therapeutic agent treatment in an eye of a subject as disclosed herein, wherein the subject has been or has not been treated with a steroid, optionally a corticosteroid, and further optionally a glucocorticosteroid, prior to the determining step.

In some embodiments, the present invention provides the method of predicting anatomical response to an anti-VEGF therapeutic agent treatment in an eye of a subject as disclosed herein, wherein the subject has been or has not been treated with an anti-inflammatory agent or immunosuppressive agent, or in combination, to the subject, optionally wherein the anti-inflammatory agent or immunosuppressive agent is selected from the group consisting of glucocorticosteroid, methotrexate, cyclosporin A, tacrolimus, azathioprine, or a biologic agent targeting a pro-inflammatory molecule such as interleukin-6, interleukin-8, and tumor necrosis factor-alpha.

In some embodiments, the present invention provides the method of predicting anatomical response to an anti-VEGF therapeutic agent treatment in an eye of a subject as disclosed herein, wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In some embodiments, the present invention provides the method of predicting anatomical response to an anti-VEGF therapeutic agent treatment in an eye of a subject as disclosed herein, wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In some embodiments, the present invention provides the method of predicting anatomical response to an anti-VEGF therapeutic agent treatment in an eye of a subject as disclosed herein, wherein the subject is a human.

In some embodiments, the present invention provides the method of predicting anatomical response to an anti-VEGF therapeutic agent treatment in an eye of a subject as disclosed herein,

In some embodiments, the present invention provides the method of predicting anatomical response to an anti-VEGF therapeutic agent treatment in an eye of a subject as disclosed herein, wherein the anti-VEGF therapeutic agent is formulated for intravitreal, suprachoroidal, and/or subretinal administration, optionally wherein the method further comprises admixing the anti-VEGF therapeutic agent and a pharmaceutically acceptable excipient or carrier.

In any of the embodiments herein, the subject may have not been treated with the anti-VEGF therapeutic agent or another anti-VEGF therapeutic agent prior to the determining step.

In any of the embodiments herein, the subject may have been treated with the anti-VEGF therapeutic agent or another anti-VEGF therapeutic agent prior to the determining step.

In any of the embodiments herein, the subject may have not been treated with a steroid, optionally a corticosteroid, and further optionally a glucocorticosteroid, prior to the determining step.

In any of the embodiments herein, the subject may have been treated with a steroid, optionally a corticosteroid, and further optionally a glucocorticosteroid, prior to the determining step.

In any of the embodiments herein, the subject may be selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

In any of the embodiments herein, the subject may be selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

In any of the embodiments herein, the subject may be a human. In any of the embodiments herein, the anti-VEGF therapeutic agent can be formulated for intravitreal, suprachoroidal, and/or subretinal administration.

In any of the embodiments herein, the method may further comprise admixing the anti-VEGF therapeutic agent and a pharmaceutically acceptable excipient or carrier.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a”, “an” and “the” comprise plural referents unless the context clearly dictates otherwise. For example, “a” or “an” means “at least one” or “one or more.”

Throughout the present disclosure, various aspects are presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the present disclosure. The upper and lower limits of these smaller ranges may independently be comprised in the smaller ranges, and are also encompassed within the present disclosure, subject to any specifically excluded limit in the stated range. Where the stated range comprises one or both of the limits, ranges excluding either or both of those comprised limits are also comprised in the present disclosure. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### REFERENCES

1. Branch Vein Occlusion Study Group. Argon laser scatter photocoagulation for prevention of neovascularization and vitreous hemorrhage in branch vein occlusion. A randomized clinical trial. Archives of ophthalmology (Chicago, Ill.: 1960), 1986. 104 (1): p. 34-41.
2. The Central Vein Occlusion Study. Baseline and early natural history report. Archives of Ophthalmology, 1993. 111 (8): p. 1087-1095.
3. Christoffersen, N. L. B. and M. Larsen, Pathophysiology and hemodynamics of branch retinal vein occlusion. Ophthalmology, 1999. 106 (11): p. 2054-2062.
4. Noma, H., K. Yasuda, and M. Shimura, Cytokines and Pathogenesis of Central Retinal Vein Occlusion. J Clin Med, 2020. 9 (11).
5. Noma, H., K. Yasuda, and M. Shimura, Cytokines and the Pathogenesis of Macular Edema in Branch Retinal Vein Occlusion. J Ophthalmol, 2019. 2019: p. 5185128.
6. Schmidt-Erfurth, U., et al., Guidelines for the Management of Retinal Vein Occlusion by the European Society of Retina Specialists (EURETINA). Ophthalmologica, 2019. 242 (3): p. 123-162.
7. Wallsh, J. O. and R. P. Gallemore, Anti-VEGF-Resistant Retinal Diseases: A Review of the Latest Treatment Options. Cells, 2021. 10 (5).
8. Rosenbaum, J. N. and P. Weisman, The Evolving Role of Companion Diagnostics for Breast Cancer in an Era of Next-Generation Omics. Am J Pathol, 2017. 187 (10): p. 2185-2198.
9. Modi, A., et al., Aqueous Humor Cytokines and Therapeutic Customization in Nonresponding Macular Edema Secondary To Retinal Vein Occlusion. Retin Cases Brief Rep, 2021. 15 (2): p. 127-130.
10. Campochiaro, P. A., et al., Ranibizumab for macular edema due to retinal vein occlusions: implication of VEGF as a critical stimulator. Mol Ther, 2008. 16 (4): p. 791-9.
11. Park, S. P. and J. K. Ahn, Changes of aqueous vascular endothelial growth factor and pigment epithelium-derived factor following intravitreal bevacizumab for macular oedema secondary to branch retinal vein occlusion. Clinical & experimental ophthalmology, 2009. 37 (5): p. 490-5.
12. Wang B, Zhang X, Chen H, Koh A, Zhao C, Chen Y, A Review of Intraocular Biomolecules in Retinal Vein Occlusion: Toward Potential Biomarkers for Companion Diagnostics. Front Pharmacol 2022. 13:859951

### EXAMPLES

The following example is included for illustrative purposes only and is not intended to limit the scope of the present disclosure.

### Example 1: Aqueous VEGF as Companion Diagnostic Marker

A magnetic particles-based chemiluminescence enzyme immunoassay (MP CLEIA) kit was developed for detection of VEGF in human aqueous humor with high sensitivity, specificity, and reproducibility. See, CN112557669A, incorporated herein by reference in its entirety for all purposes.

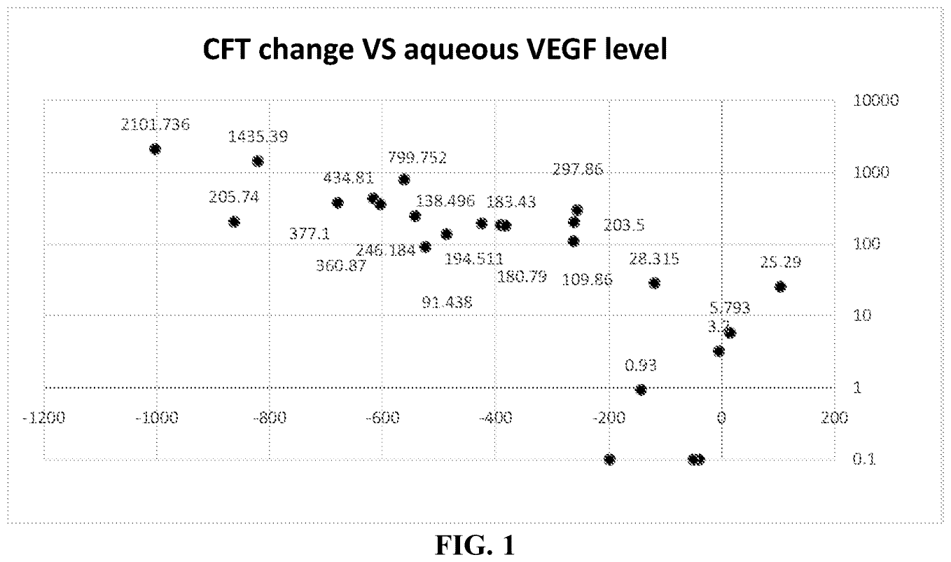

In order to test the use of aqueous VEGF as a companion biomarker for anti-VEGF treatments in RVO, aqueous humor samples were collected in 24 RVO-ME cases just before intravitreal injection of anti-VEGF agents. Changes of central macular thickness (CMT) or central foveal thickness (CFT), which is defined as the vertical distance between the internal limiting membrane and the retinal pigment epithelium at the foveal center, or maximum paravoveal retinal thickness (MRT) when fovea was not involved 2 to 4 weeks after the intravitreal injection as compared to baseline were plotted against baseline aqueous VEGF level in FIG. 1. A generally negative correlation was found between CFT/MRT change and baseline aqueous VEGF level (Spearman's rank correlation coefficient test, p<0.01). In general, higher baseline aqueous VEGF was associated with better treatment response.

Eyes with aqueous VEGF level above a cut-off value between about 10 and about 100 μg/mL had significantly better anatomical treatment response as measured by CFT change than those with lower aqueous VEGF levels. For example, FIG. 1 shows aqueous VEGF levels were below 30 μg/mL in eyes with CFT/MRT change ≥−200 μm, and aqueous VEGF levels were above 90 μg/mL in eyes with CFT/MRT change ≤−200 μm. For instance, a determined VEGF level of about 100 μg/mL can be associated with and/or used to predict a change of CFT between about-250 μm and about 0 μm, and a determined VEGF level between about 1 μg/mL and about 10 μg/mL can be associated with and/or used to predict a change of CFT between about-200 μm and about 0 μm. As such, a cut-off value of aqueous VEGF between about 10 and about 100 μg/mL can be used to divide eyes into two groups with different anatomical response. In other words, detection of base aqueous VEGF level (e.g., with the MPs-CLEIA kit described in CN112557669A or any other suitable kit) may be used to predict anatomical treatment response to anti-VEGF in RVO-ME patients as a companion diagnostic.

### Example 2: Baseline Aqueous VEGF Level and Central Macular Thickness Predict Anatomical Responses to Anti-VEGF Therapy in RVO-ME

To investigate whether aqueous biological biomarkers and anatomical imaging biomarkers have predictive value on treatment response of anti-VEGF therapy in retinal vein occlusion associated-macular edema (RVO-ME) patients, a retrospective observational study was performed in 2 tertiary-referral-centers.

### Participants

This is a dual-center observational study. Medical records of RVO-ME patients who underwent intravitreal injection of anti-VEGF agents with concomitant aqueous tap in Peking Union Medical College Hospital and Xi'an People's Hospital between March 2020 and January 2022 were reviewed. Patients were excluded if: (1) baseline (bl) (≤2 weeks before the intravitreal injection) OCT scans centered on the foveola were not available, or the quality of the bl-OCT scans was not satisfactory; (2) ME did not involve the foveola, or central macular thickness (CMT) was ≤250 μm on bl-OCT; (3) had a history of or was complicated with diabetes, retinal vasculitis, uveitis, age-related macular degeneration, high myopia, epiretinal membrane, or other fundus diseases that might cause ME; (4) had undergone intravitreal or periocular corticosteroid injection.

### Clinical Evaluations

All patients underwent a comprehensive ophthalmic examination within 2 weeks before the intravitreal injection, including best corrected visual acuity (BCVA), intraocular pressure (IOP), slit-lamp examination, funduscopic examination and OCT. Raster or radial OCT scans centered on the foveola were obtained using one of the following OCT systems: Triton, TOPCON Inc, Japan; VanGogh, SVision Imaging Inc, China; RTVue XR Avanti, Optovue, Inc, USA. CMT (thickness of retina at the foveola) was measured with the built-in software.

### Intravitreal Injection and Aqueous Tap

Antibiotics (0.5% levofloxacin) eye drops were administered 4 times per day for at least 3 days prior to the treatment. After surface anesthesia and disinfection of the conjunctiva sac with 5% povidone-iodine for 30 seconds, about 50 to 100 ul aqueous humor was collected by a 25-gauge needle syringe, followed by intravitreal injection of 50 ul of ranibizumab (0.5 mg) (Novartis, Swiss), conbercept (0.5 mg) (Chengdu Kanghong, China), or aflibercept (2 mg) (Bayer, Germany). After the injection, antibiotic ointment was applied, and the eye was patched for at least 3 hours.

### Aqueous Humor Testing

Concentrations of VEGF, IL-6, IL-8, IL-10, MCP-1 and IP-10 in aqueous humor was detected by the Luminex xMAPTM platform (Luminex® 100 system, Luminex corporation, USA) using Magnetic Luminex® Performance Assay kits (R&D Systems Inc., USA), following the manufacturers' instructions.

The MPCLEIA kit was used to measure the aqueous VEGF level. This kit requires only 20 ul of aqueous humor samples with limit of blank (LOB) of about 2 μg/ml, limit of detection (LOD) of about 5 μg/ml, and linear range of about 5 to 5000 μg/ml. The intra-assay and inter-assay coefficient of variations (CV) are about 10% and 15%, respectively.

### Subgroup Evaluation of Treatment Responses

RVO eyes were divided into 2 groups based on history of treatment. Anti-VEGF treatment-naive eyes and those with previous anti-VEGF treatment more than 3 months before were designated as the non-treated group; and eye with previous anti-VEGF treatment within 3 months were classified into the treated group.

### Definition of Treatment Response

Eyes with complete clinical evaluation 2-6 weeks after the injection were divided into 2 groups in terms of their anatomical treatment response (ATR) to the intravitreal treatment. ATR was indicated by relative CMT change after the intravitreal injection (ΔCMT/bl-CMT) and categorized as good response (GR) (≤-25%) and poor response (PR) (>-25%).

### Data Analysis

Statistical analyses were processed by IBM SPSS statistics 26.0 (SPSS Inc, Chicago, IL), RStudio version 1.4.1717 (RStudio Team 2021) or GraphPad Prism 8 (GraphPad Prism Software Inc., San Diego, CA). Measurements below detection limit was replaced by 30% of the LOD value of the detection platform for statistical analysis without changing the rank of the included data. Unmeasured samples were processed as missing values which were not included in data analysis.

Normally and non-normally distributed continuous variables were presented as means±standard deviations (SD) and median (inter-quartile range (IQR)), respectively; discrete variables were presented as counts (%). Comparisons of unpaired data were performed using unpaired Student's t-test for normal distributed data, non-parametric Mann-Whitney U test for non-normal distribution data, and Chi-square test, Fisher or Wilcoxon test for discrete variables.

Receiver operating characteristic (ROC) curves were generated to assess the predictive efficacy of different variables and regression models on ATR. Optimal cut-off values were determined as the points at which the Youden index (sensitivity+specificity-1) was maximal. The prognostic factors were investigated using univariate and multivariate logistic regression models.

Univariate and multivariate logistic regression analyses were conducted to assess factors associated with ATR with the Wald test. The automatic stepwise regression developed a sequence of logistic regression models to work on the variable selection with each step adding or dropping a covariate. The procedure of stepwise regression examined deviance value and Akaike Information Criterion (AIC) for each variable in each step. A preferred model was determined by removing a variable with highest AIC value in each step. The likelihood-ratio test (LRT) was performed to compare the goodness of fit for two nested models. A p-value<0.05 was considered statistically significant.

### Results

Ninety-four patients (44 females and 50 males) (94 eyes) with a mean age of 53.12±15.67 years old were included in this study. Of the 94 eyes with RVO-ME, 41 were BRVO and 53 were CRVO; 80, 7 and 7 eyes were treated with ranibizumab (IVR), aflibercept (IVA) and conbercept (IVC), respectively. Patients were categorized into the non-treated and treated groups as described previously, which included 58 and 36, respectively. The general demographic and clinical features of the non-treated (with minimal or no influence of previous anti-VEGF therapy) and treated groups are presented in Table 1.

TABLE 1
General demographic and clinical features of the included patients

| | Non-treated | Treated | t/χ²/z | p |
|---|---|---|---|---|
| Patients (n) | 58 | 36 | — | — |
| Mean age at diagnosis | 54.52 ± 15.52 | 50.86 ± 15.87 | 1.100 | 0.274 |
| (years) | | | | |
| Female (n (%)) | 23(40) | 13(36) | 0.118 | 0.731 |
| Left eye (n (%)) | 28(48) | 9(25) | 5.042 | 0.025* |
| BRVO (n (%)) | 26(48) | 15(44) | 0.136 | 0.712 |
| IVR/IVC/IVA (n (%)) | 47(81)/6(10)/5(9) | 32(91)/1(3)/2(6) | 2.283 | 0.319 |
| Previous laser therapy (n | 11(19) | 5(14) | 0.405 | 0.524 |
| (%)) | | | | |
| Course of disease | 9.70 ± 18.03 | 6.87 ± 6.85 | 1.223 | 0.221 |
| bl-BCVA (LogMAR) | 1.01 ± 0.55 | 0.89 ± 0.46 | −0.677 | 0.498 |
| b1-CMT (μm) | 630.13 ± 312.86 | 312.17 ± 143.63 | −5.040 | <0.001* |

Abbreviations: BRVO, branch retinal vein occlusion; IVR/IVC/IVA, intravitreal ranibizumab/conbercept/aflibercept; bl, baseline; BCVA, best corrected visual acuity; LogMAR, logarithm of the minimum angle of resolution; CMT, central macular thickness.
*P < .05

### Analyses of Aqueous Cytokines

Aqueous concentrations of IL-6, IL-8, IL-10, IP-10, MCP-1, and VEGF in all 94 included eyes were 13.8 (5.1-31.5), 52.5 (22.8-107.8), 2.3 (0.5-5.2), 65.5 (22.9-106.6), 1345.9 (779.2-1745.4), and 75.6 (5.8-204.1), respectively. Correlation analyses revealed an extensive association among cytokines/measurements. In particular, VEGF level was significantly correlated to all other cytokines/measurements; among all the cytokines, IL-10 was the least associated with other cytokines.

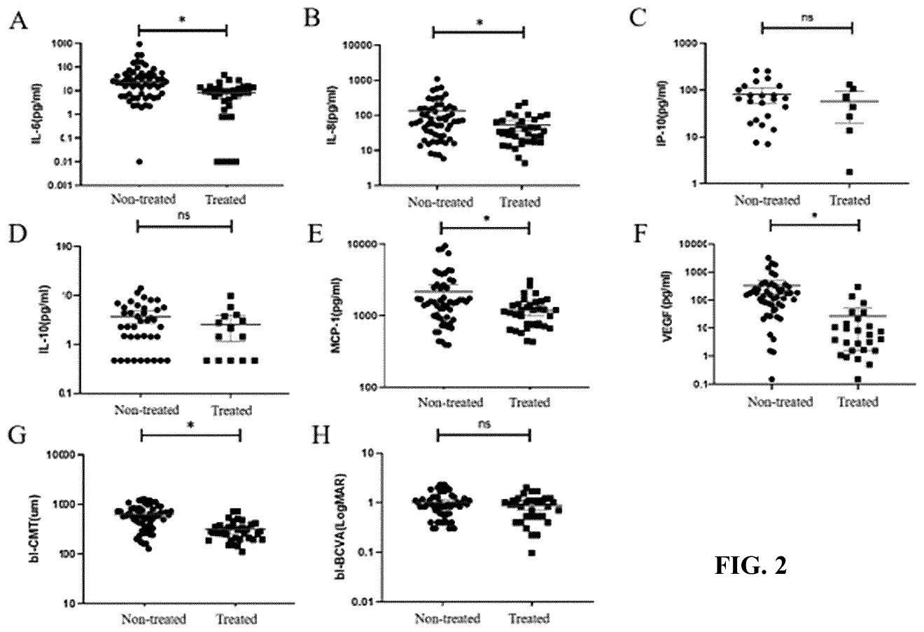

Subgroup analyses revealed that aqueous VEGF (5.23 (1.56-14.49) versus 151.44 (62.17-297.86) pg/ml), IL-6 (8.35 (1.57-13.84) versus 22.00 (6.00-51.84) pg/ml), IL-8 (34.39 (18.84-70.97) versus 69.31 (26.26-156.78) pg/ml), and MCP-1 (1150.73 (731.81-1442.02) versus 1540.65 (936.36-2594.05) pg/ml) in the treated group were significantly lower than those in the non-treat group (Z=−4.83, P<0.001; Z=−5.43, P<0.001; Z=−4.07, P<0.001; Z=−2.57, P=0.010; Z=−2.62, P=0.009; respectively). There was a significantly higher level of bl-CMT (593 (390-824.5) versus 284 (197-376) μm) in the non-treated group versus the treated group, but no significant difference in bl-BCVA (logarithm of the minimum angle of resolution (logMAR) scale) was found between the two groups (Z=−4.97, P<0.001; Z=−0.79, P=0.432; respectively) (FIG. 2).

Identification of Aqueous Cytokines Associated with Treatment Response

Among the 94 included patients, follow up data 2-6 weeks after the injection was available in 40 patients with IVR. Of these 40 IVR patients, 31 and 9 patients respectively were categorized into the GR and PR groups based on ATR (ΔCMT/bl-CMT).

Correlation analysis was conducted in order to identify aqueous cytokines that may have predictive value on treatment response, and revealed that ΔCMT/bl-CMT was significantly associated with aqueous IL-8 (r=−0.330, P=0.040), and VEGF (r=−0.506, P=0.001), but not with IL-6 (r=−0.312, P=0.053), IP-10 (r=−0.259, P=0.298), IL-10 (r=−0.081, P=0.698), and MCP-1 (r=−0.270, P=0.101).

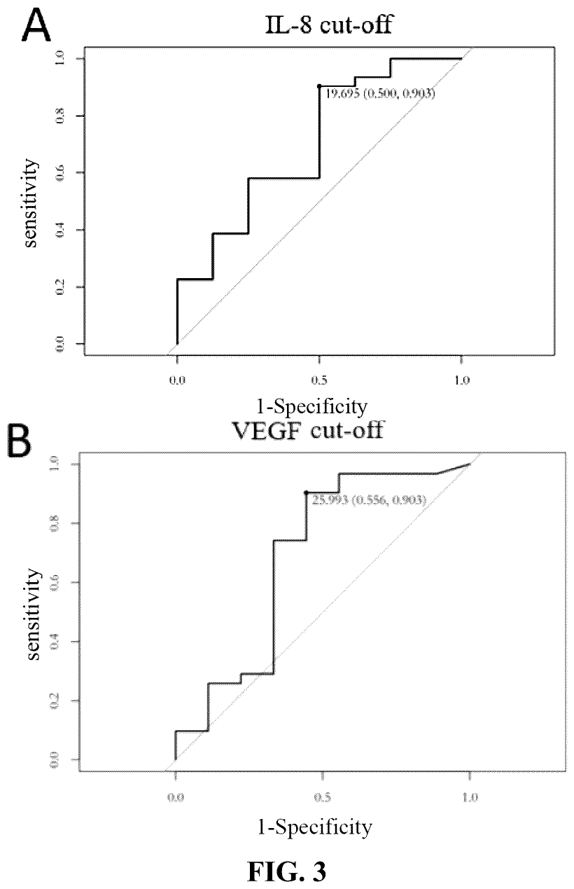

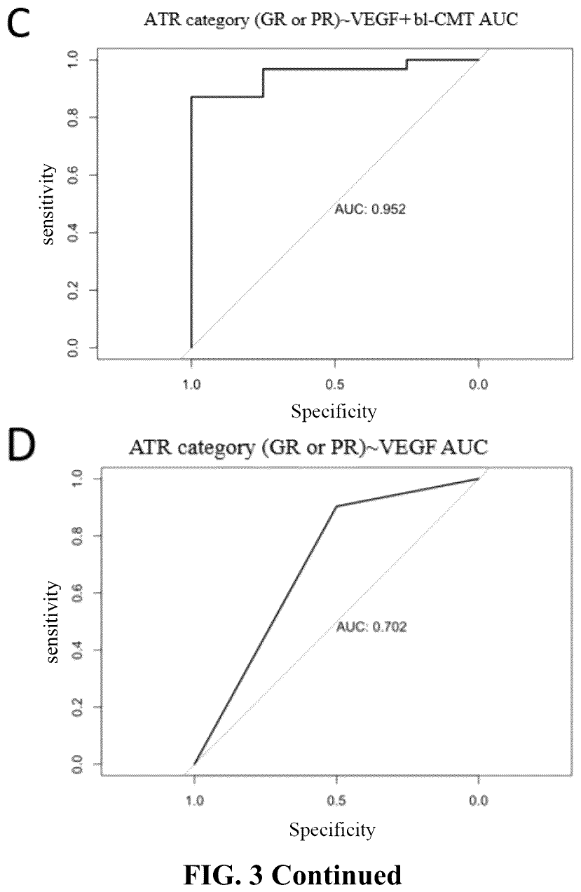

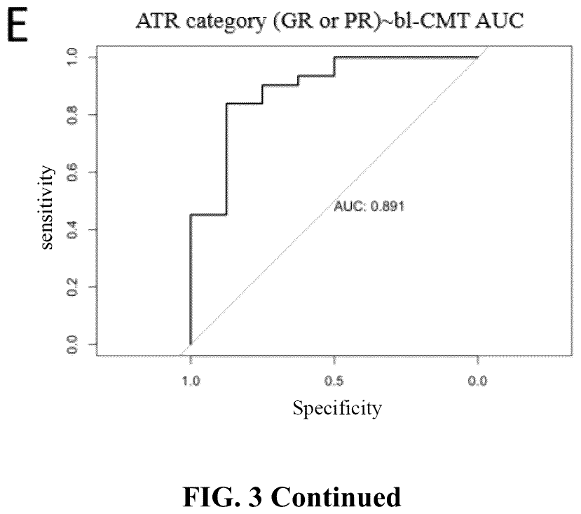

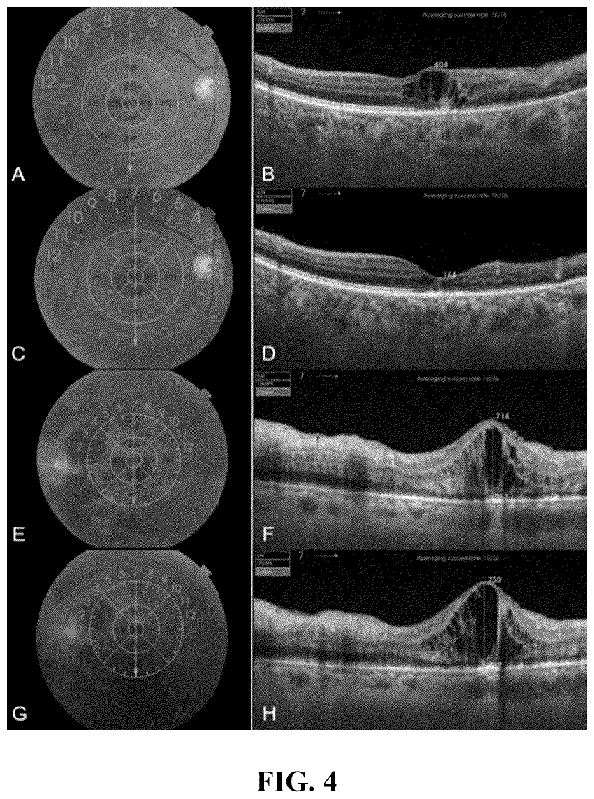

ROC analysis with Youden's J index identified optimal cut-off values of 19.7 pg/ml and 26.0 pg/ml for IL-8 and VEGF, respectively (FIG. 3). Accordingly, 28/3, 25/6 and 28/3 of the 31 patients in the GR group, and 4/4 (one missing value for IL-8 data), 4/5 and 4/5 of the 9 patients in the PR group, had high (≥cut-off value)/low (<cut-off value) levels of aqueous IL-8 (n=39), and VEGF (n=40), respectively. Chi-square test revealed significant associations between ATR category (GR or PR) and aqueous IL-8, and VEGF level (high or low) (χ²=7.020, P=0.008; χ²=11.176, P<0.001; respectively).

Wilcoxon-Mann-Whitney U test also revealed significant differences in data distribution of ATR (ΔCMT/bl-CMT) between eyes with high and low IL-8 (W=48, P=0.017), and VEGF (W=55, P=0.012) levels. Both Chi-square and Wilcoxon-Mann-Whitney U tests confirmed that categorization of patients based on the ROC derived cut-off values was reasonable.

Univariate analysis further confirmed that ATR (ΔCMT/bl-CMT) was significantly associated with aqueous IL-8 (z=2.396, P=0.017), and VEGF (z=2.715, P<0.001) (Table 2).

TABLE 2
Univariate and multivariate logistic regression analysis

| | | | |
|---|---|---|---|
| Estimate | Standard error | z-value | P value |

Univariate

| | | | | |
|---|---|---|---|---|
| IL-8 | 2.234 | 0.932 | 2.396 | 0.017* |
| VEGF | 2.457 | 0.905 | 2.715 | <0.001*** |

Multivariate
Model: ATR category ~ VEGF + bl-CMT

| | | | | |
|---|---|---|---|---|
| Intercept | −8.260 | 3.421 | −2.415 | 0.016* |
| VEGF ^{a} | 3.939 | 1.867 | 2.110 | 0.035* |
| bl-CMT | 0.013 | 0.005 | 2.503 | 0.012* |

Abbreviations: IL, interleukin; bl-CMT, baseline central macular thickness.
*P < .05;
**P < .01;
***P < .001.
^{a} Low level of VEGF was defined as reference.

### Establishment and Validation of the Optimal Predictive Model for Treatment Response

In order to establish an optimal predictive model of treatment response, a multivariate logistic stepwise regression analysis using variates including IL-8 (high or low), VEGF (high or low), bl-CMT, the duration of the disease, treatment history (IVR non-treated or treated, whether underwent laser photocoagulation before), disease phenotype (CRVO or BRVO). After stepwise screening and LRT comparison, variates VEGF and bl-CMT remained in the model with lower AIC. In addition, bl-CMT and VEGF were significantly correlated with ATR category in multivariate logistic regression analysis (Table 2).

The predictive efficacy of the above-mentioned univariate and multivariate logistic regression models were further assessed by ROC curve analysis and LRT. As a result, AUCs was 0.952 for multivariate logistic regression models of ATR category ˜VEGF+bl-CMT, comparing to 0.702 and 0.891 for univariate logistic regression models of VEGF and bl-CMT, respectively (FIG. 3). According to LRT results, the logistic regression model combining VEGF with bl-CMT showed a higher Goodness-of-Fit than any models with VEGF (Deviance (D)=−16.18, P<0.001) or bl-CMT (D=−7.596, P=0.006) alone. These results suggest that aqueous biomarkers (e.g. VEGF) and imaging markers (e.g. bl-CMT) could be integrated to improve accuracy in predicting anatomical response to anti-VEGF therapeutic agent treatment in eye(s) of a subject.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-14. (canceled)

15. A kit comprising i) an anti-VEGF therapeutic agent and ii) instructions of administering the anti-VEGF therapeutic agent to a subject having a level of VEGF in the aqueous humor higher than a cut-off value, wherein the cut-off value is between 10 and 100 μg/mL, or wherein the kit comprises i) a reagent for determining a level of VEGF in the aqueous humor of a subject, and ii) instructions comprising a cut-off value of VEGF level and optionally instructions of comparing the determined level of VEGF to the cut-off value, wherein the cut-off value is between 10 and 100 μg/mL.

16-28. (canceled)

29. A composition comprising i) isolated aqueous humor from a subject, and ii) a reagent that directly or indirectly interacts with VEGF in the isolated aqueous humor, wherein the isolated aqueous humor comprises a level of VEGF higher or lower than a cut-off value, and the cut-off value is between 10 and 100 μg/mL.

30-45. (canceled)

46. A method for predicting anatomical response to an anti-VEGF therapeutic agent treatment in a subject's eye, comprising determining a VEGF level in the aqueous humor collected from the subject, wherein a determined VEGF level higher than a cut-off value indicates that the eye has a good anatomical response to the anti-VEGF therapeutic agent treatment, wherein a determined VEGF level no higher than the cut-off value indicates that the eye has a poor anatomical response to the anti-VEGF therapeutic agent treatment, and wherein the cut-off value is between 10 and 100 μg/mL.

47-71. (canceled)

72. A method for predicting anatomical response to an anti-VEGF therapeutic agent treatment in an eye of a subject, comprising determining the VEGF level in the aqueous humor collected from the subject and the bl-CMT level in the subject,
wherein the determined VEGF level higher than the VEGF cut-off value and the determined bl-CMT level higher than the bl-CMT cut-off value indicate that the eye has a good anatomical response to the anti-VEGF therapeutic agent treatment; and
wherein the determined VEGF level no higher than the VEGF cut-off value or the determined bl-CMT level no higher than the bl-CMT cut-off value indicate that the eye has a poor anatomical response to the anti-VEGF therapeutic agent treatment.

73. The method of claim 72, wherein the VEGF cut-off value is between 10 and 100 μg/mL.

74. The method of claim 72, wherein the bl-CMT cut-off value is between about 250 and about 600 μm.

75. The method of claim 72, wherein the anatomical response to the anti-VEGF therapeutic agent is measured by change of CMT which is defined as the vertical distance between the internal limiting membrane and the retinal pigment epithelium at the foveal center.

76. The method of claim 72, wherein the anatomical response to the anti-VEGF therapeutic agent is measured by relative change of CMT, wherein the relative change of CMT is defined by change of CMT divided by bl-CMT.

77. The method of claim 72, wherein the determined VEGF level higher than a VEGF cut-off value of between 10 and 100 μg/mL and the determined bl-CMT level higher than a bl-CMT cut-off value of between about 250 and about 600 μm indicate that the eye has good anatomical response to the anti-VEGF therapeutic agent treatment.

78. The method of claim 72, wherein the determined VEGF level higher than the VEGF cut-off value and the determined bl-CMT level higher than the bl-CMT cut-off value predict a change of CFT≤200 μm.

79. The method of claim 72, wherein the determined VEGF level lower than the VEGF cut-off value or the determined bl-CMT level lower than the bl-CMT cut-off value predicts a change of CFT≥200 μm.

80. The method of claim 72, wherein the VEGF cut-off value is between 30 and 90 μg/mL.

81-84. (canceled)

85. The method of claim 72, wherein the subject is in need of treatment of retinal vein occlusion (RVO), optionally wherein the retinal vein occlusion is central retinal vein occlusion (CRVO) or branch retinal vein occlusion (BRVO).

86. The method of claim 72, wherein the subject is in need of treatment of macular edema secondary to retinal vein occlusion (ME-RVO), optionally wherein the retinal vein occlusion is central retinal vein occlusion (CRVO) and/or branch retinal vein occlusion (BRVO).

87. The method of claim 72, further comprising administering the anti-VEGF therapeutic agent or another anti-VEGF therapeutic agent intravitreally, suprachoroidally, and/or subretinally to the subject when the determined VEGF level is higher 90 pg/mL and when the determined bl-CMT level is higher than the bl-CMT cut-off value.

88-89. (canceled)

90. The method of claim 72, further comprising administering a therapy other than the anti VEGF therapeutic agent steroid, an anti-inflammatory agent, and/or an immunosuppressive agent to the subject when the determined VEGF level is lower than 30 pg/mL.

91-92. (canceled)

93. The method of claim 72, wherein the subject has not been treated with the anti-VEGF therapeutic agent or another anti-VEGF therapeutic agent prior to the determining step.

94. The method of claim 72, wherein the subject has been treated with a steroid an anti-inflammatory agent, and/or an immunosuppressive agent, prior to the determining step.

95-96. (canceled)

97. The method of claim 72, wherein the determined VEGF level is higher than 10-100 μg/mL and the determined bl-CMT level is higher than 250-600 μm, and
wherein the subject is selected to initiate, continue, or resume treatment with the anti-VEGF therapeutic agent or another anti-VEGF therapeutic agent, and/or selected to discontinue treatment with a steroid, optionally a corticosteroid, and further optionally a glucocorticosteroid.

98. The method of claim 72, wherein the determined VEGF level is lower than 10-100 μg/mL and/or the determined bl-CMT level is lower than 250-600 μm, and
wherein the subject is selected to discontinue treatment with the anti-VEGF therapeutic agent or another anti-VEGF therapeutic agent, and/or selected to initiate, continue, or resume treatment with a steroid, optionally a corticosteroid, and further optionally a glucocorticosteroid.

99-100. (canceled)
